SEQUENCE LISTING (17 sequences)

SEQ ID NO: 1 - protein, 22 aa, Artificial Sequence
  source: /note=“Description of Artificial Sequence Synthetic peptide”
DYKDHDGDYKDHDIDYKDDDDK

SEQ ID NO: 2 - protein, 7 aa, Simian virus 40
PKKKRKV

SEQ ID NO: 3 - DNA, 28 bp, Artificial Sequence
  source: /note=“Description of Artificial Sequence Synthetic oligonucleotide”
aaagcaactgttagcttgcactagacta

SEQ ID NO: 4 - DNA, 28 bp, Artificial Sequence
  source: /note=“Description of Artificial Sequence Synthetic oligonucleotide”
cacaggctccaggaagggtttggcctct

SEQ ID NO: 5 - protein, 7 aa, Artificial Sequence
  source: /note=“Description of Artificial Sequence Synthetic peptide”
DQSNLRA

SEQ ID NO: 6 - protein, 7 aa, Artificial Sequence
  source: /note=“Description of Artificial Sequence Synthetic peptide”
RNFSLTM

SEQ ID NO: 7 - protein, 7 aa, Artificial Sequence
  source: /note=“Description of Artificial Sequence Synthetic peptide”
STGNLTN

SEQ ID NO: 8 - protein, 7 aa, Artificial Sequence
  source: /note=“Description of Artificial Sequence Synthetic peptide”
TSGSLTR

SEQ ID NO: 9 - protein, 7 aa, Artificial Sequence
  source: /note=“Description of Artificial Sequence Synthetic peptide”
AQCCLFH

SEQ ID NO: 10 - protein, 7 aa, Artificial Sequence
  source: /note=“Description of Artificial Sequence Synthetic peptide”
RNDHRTT

SEQ ID NO: 11 - protein, 7 aa, Artificial Sequence
  source: /note=“Description of Artificial Sequence Synthetic peptide”
QKAHLIR

SEQ ID NO: 12 - protein, 7 aa, Artificial Sequence
  source: /note=“Description of Artificial Sequence Synthetic peptide”
QKGTLGE

SEQ ID NO: 13 - protein, 7 aa, Artificial Sequence
  source: /note=“Description of Artificial Sequence Synthetic peptide”
RGRDLSR

SEQ ID NO: 14 - protein, 7 aa, Artificial Sequence
  source: /note=“Description of Artificial Sequence Synthetic peptide”
RRDNLHS

SEQ ID NO: 15 - RNA, 1727 nt, Artificial Sequence
  source: /note=“Description of Artificial Sequence Synthetic polynucleotide”
gggagacaagcuuugaauuacaagcuugcuuguucuuuuugcagaagcucagaauaaacgcucaacuuuggcagaucgaauucgccauggacuacaaagaccaugacggugauuauaaagaucaugacaucgauuacaaggaugacgaugacaagauggcccccaagaagaagaggaaggucggcauccacgggguacccgccgcuauggcugagaggcccuuccagugucgaaucugcaugcagaacuucagugaccaguccaaccugcgcgcccacauccgcacccacaccggcgagaagccuuuugccugugacauuugugggaggaaauuugcccgcaacuucucccugaccaugcauaccaagauacacacgggcagccaaaagcccuuccagugucgaaucugcaugcagaacuucaguuccaccggcaaccugaccaaccacauccgcacccacaccggcgagaagccuuuugccugugacauuugugggaggaaauuugccaccuccggcucccugacccgccauaccaagauacacacgcacccgcgcgccccgaucccgaagcccuuccagugucgaaucugcaugcagaacuucagugaccaguccaaccugcgcgcccacauccgcacccacaccggcgagaagccuuuugccugugacauuugugggaggaaauuugccgcccaguguugucuguuccaccauaccaagauacaccugcggggauccaucagcagagccagaccacugaacccgcacccggagcuggaggagaagaaguccgagcugcggcacaagcugaaguacgugccccacgaguacaucgagcugaucgagaucgccaggaacagcacccaggaccgcauccuggagaugaaggugauggaguucuucaugaagguguacggcuacaggggaaagcaccugggcggaagcagaaagccugacggcgccaucuauacagugggcagccccaucgauuacggcgugaucguggacacaaaggccuacagcggcggcuacaaucugccuaucggccaggccgacgagauggagagauacguggaggagaaccagacccgggauaagcaccucaaccccaacgagugguggaagguguacccuagcagcgugaccgaguucaaguuccuguucgugagcggccacuucaagggcaacuacaaggcccagcugaccaggcugaaccacaucaccaacugcaauggcgccgugcugagcguggaggagcugcugaucggcggcgagaugaucaaagccggcacccugacacuggaggaggugcggcgcaaguucaacaacggcgagaucaacuucagaucuugauaacucgagucuagaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcgcuagaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcgggacauucuuaauuaaaaaaaaaaaaaaaaaaaaaaaaaaaaaaaaaaaaaaaaaaaaaaaaaaaaaaaaaaaacuag

SEQ ID NO: 16 - RNA, 1680 nt, Artificial Sequence
  source: /note=“Description of Artificial Sequence Synthetic polynucleotide”
gggagacaagcuugaauacaagcuugcuuguucuuuuugcagaagcucagaauaaacgcucaacuuuggcagaucgaauucgccuagagaucuggcggcggagagggcagaggaagucuucuaaccugcggugacguggaggagaaucccggcccuaggaccauggacuacaaagaccaugacggugauuauaaagaucaugacaucgauuacaaggaugacgaugacaagauggcccccaagaagaagaggaaggucggcauucaugggguacccgccgcuauggcugagaggcccuuccagugucgaaucugcaugcagaaguuugcccgcaacgaccaccgcaccacccauaccaagauacacacgggcgagaagcccuuccagugucgaaucugcaugcagaacuucagucagaaggcccaccugauccgccacauccgcacccacaccggcgagaagccuuuugccugugacauuugugggaggaaauuugcccagaagggcacccugggcgagcauaccaagauacacacgggaucucagaagcccuuccagugucgaaucugcaugcagaacuucagucgcggccgcgaccugucccgccacauccgcacccacaccggcgagaagccuuuugccugugacauuugugggaggaaauuugcccgccgcgacaaccugcacucccauaccaagauacaccugcggggaucccagcuggugaagagcgagcuggaggagaagaaguccgagcugcggcacaagcugaaguacgugccccacgaguacaucgagcugaucgagaucgccaggaacagcacccaggaccgcauccuggagaugaaggugauggaguucuucaugaagguguacggcuacaggggaaagcaccugggcggaagcagaaagccugacggcgccaucuauacagugggcagccccaucgauuacggcgugaucguggacacaaaggccuacagcggcggcuacaaucugccuaucggccaggccgacgagaugcagagauacgugaaggagaaccagacccggaauaagcacaucaaccccaacgagugguggaagguguacccuagcagcgugaccgaguucaaguuccuguucgugagcggccacuucagcggcaacuacaaggcccagcugaccaggcugaaccgcaaaaccaacugcaauggcgccgugcugagcguggaggagcugcugaucggcggcgagaugaucaaagccggcacccugacacuggaggaggugcggcgcaaguucaacaacggcgagaucaacuucugauaacucgagucuagaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcgcuagaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcgggacauucuuaauuaaaaaaaaaaaaaaaaaaaaaaaaaaaaaaaaaaaaaaaaaaaaaaaaaaaaaaaaaaaacuag

SEQ ID NO: 17 - protein, 9 aa, Unknown
  source: /note=“Description of Unknown ′LAGLIDADG′ family peptide motif sequence”
LAGLIDADG

(19) United States
(12) Patent Application Publication
(10) Pub. No.: US 2020/0316116 A1
(43) Pub. Date: Oct. 8, 2020

(54) METHOD FOR THE TREATMENT OF BETA-THALASSEMIA

(71) Applicant: Sangamo Therapeutics, Inc., Richmond, CA (US)

(72) Inventors: Weston P. Miller, IV, Richmond, CA (US); John Tomaro, Richmond, CA (US); Sagar A. Vaidya, Richmond, CA (US); Mark Walters, Oakland, CA (US)

(21) Appl. No.: 16/835,776

(22) Filed: Mar. 31, 2020

Related U.S. Application Data
(60) Provisional application No. 62/828,182, filed on Apr. 2, 2019.
(60) Provisional application No. 62/930,846, filed on Nov. 5, 2019.
(60) Provisional application No. 62/944,626, filed on Dec. 6, 2019.

(51) Int. Cl. A61K 35/12; C12N 5/078; A61P 7/00

(52) U.S. Cl. CPC A61K 35/12; A61K 31/255; A61P 7/00; C12N 5/0634

(57) ABSTRACT

Described herein are methods and compositions for treating a beta-thalassemia. p

### CROSS-REFERENCE TO RELATED APPLICATIONS

The present application claims the benefit of U.S. Provisional Application No. 62/828,182, filed Apr. 2, 2019; U.S. Provisional Application No. 62/930,846, filed Nov. 5, 2019; and U.S. Provisional Application No. 62/944,626, filed Dec. 6, 2019, the disclosures of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Jan. 10, 2020, is named 8328-0194_SL.txt and is 8,698 bytes in size.

### TECHNICAL FIELD

The present invention concerns methods for treating β-thalassemia, and gene therapy.

### BACKGROUND

β-thalassemia is an inherited anemia characterized by absent or defective β-globin chain synthesis (Higgs & Engel (2012) Lancet 379(9813):373-83). The defect causes an imbalance in globin chain production, and a reduction in hemoglobin (which is made up of two α-globin and two β-globin chains). As a consequence of the globin chain imbalance, unstable α-globin chain tetramers form in red blood cells (RBCS) or RBC precursors, and intramedullary destruction, apoptosis, ineffective erythropoiesis, iron overload, and profound anemia, occur (Origa, R. (2017) Genet Med 19(6):609-619).

The thalassemias (β and α) are the most common monogenic diseases in man. They have a worldwide distribution, but are most common in South Asia, the Indian subcontinent, the Middle East and Mediterranean regions, and sub-Saharan Africa (Modell et al. (2008) J Cardiovasc Magn Reson. 10:42; Colah et al. (2010) Expert Rev Hematol 3(1):103-17). It is estimated that about 1.5% of the global population are carriers of a β-thalassemia mutation (e.g., a G→C mutation at nucleotide 5 of the IVS-I “IVS-I-5”; a C>T mutation at nucleotide 654 of IVS-II “IVS-II-654’, with about 60,000 symptomatic individuals born each year (Galanello & Origa (2010) Orphanet J Rare Dis. 5:11).

The clinical severity of β-thalassemia is determined by the amount of normal hemoglobin produced, and defines three clinical and hematological conditions, classically referred to as β-thalassemia minor, β-thalassemia intermedia, and β-thalassemia major. Patients with β-thalassemia minor have mild or no anemia, and are usually asymptomatic carriers. Patients with β-thalassemia intermedia have a moderately severe anemia, and may benefit from transfusions to improve their quality-of-life, but later in life often develop a transfusion-dependent phenotype. Patients with β-thalassemia major have a severe anemia and require frequent blood transfusions for life. Morbidities resulting from the anemia include failure to thrive, skeletal deformities, pulmonary hypertension, venous thromboembolism, liver cirrhosis, heart failure, leg ulcers, and endocrine dysfunction (Vichinsky et al. (2005) Pediatrics. 116(6):e818-25). Although there are many combinations of β-globin mutations and genetic disease modifiers that are associated with the transfusion-dependent phenotype, collectively the condition is referred to in this study as transfusion-dependent β-thalassemia (TDT) (Galanello & Origa, ibid).

Improvements in health outcomes for patients with TDT have occurred over the past 50 years as the benefits of a supportive care program became recognized. The program consists of regular RBC transfusions, starting as soon as the diagnosis is established and anemia develops. The RBC transfusions are accompanied by regular iron chelation therapy to reduce the iron overload in vital organs that is caused by the transfusions. This supportive care program significantly meliorates the morbidity of TDT, however even with this program, 20% of treated patients having a life expectancy of less than 40 years (Modell et al. (2008) Cardiovasc Magn Reason 10:42). In addition, the program is time-consuming and resource-intense where treatment of a single patient for 50 years was estimated in 2011 to cost $1,971,380 USD (Koren et al. (2014) Mediterr J Hematol Infect Dis 6(1): e2014012).

Currently, the only proven cure for TDT is allogeneic hematopoietic stem cell transplantation (HSCT). Allogeneic HSCT carries substantial risk of chronic morbidity (e.g., graft-versus-host disease [GVHD]) as well as a 10-15% risk of death based on 5-year mortality (Locatelli et al. (2013) Blood 122(6):1072-8; Baronciani et al. (2016) Bone Marrow Transplant 51(4):536-41). In addition, published reports show that the probability of identifying a well-matched unrelated allogeneic donor is influenced by the ethnicity of the recipient; for example, among individuals of African descent, the probability of finding a suitable donor is estimated to be only 19% (Gragert et al. (2014) N Engl J Med. 371(4):339-48). Thus, many, if not most, recipients will lack a human leukocyte antigen (HLA)-matched donor for allogeneic HSCT, making this potential curative treatment unavailable.

Thus, there remains a need for compositions and methods for treating and/or preventing TDT.

### SUMMARY

Disclosed herein are compositions and methods for treating and/or preventing β-thalassemia in a subject in need thereof. The present disclosure provides methods and compositions for genome editing and/or gene transfer. The present disclosure also provides methods and compositions for cell therapy for the treatment of subjects lacking sufficient expression of beta globin. (e.g., β0/β0 or non-β0/β0 subjects). Aberrant beta globin expression in the subject may be caused by any mutation, including but not limited to one or more of the following mutations: IVS-I-5; IVS-II-654. In some embodiments, the methods and compositions disclosed herein are used to treat transfusion-dependent β-thalassemia (TDT). The disclosure provides methods of treating a subject with β-thalassemia comprising administering cells that have been modified using engineered nucleases to the subject wherein the subject is treated. Cells administered to the patient may be autologous (isolated from the patient, genetically modified and then reinfused into the patient) or allogenic cells, for example isolated from healthy patients and infused into the patient.

Methods of altering expression of hemoglobin, including for use in the treatment of TDT, as provided herein, include methods that result in a change from baseline of clinical laboratory hemoglobin fractions (adult hemoglobin, HbA and fetal hemoglobin, HbF) in terms of both changes in grams/dL plasma and percent HbF of total Hb in a subject. In some embodiments, use of the methods of treatment disclosed herein may result in a change of thalassemia-related disease biomarkers. In some embodiments, changes in the thalassemia-related disease biomarkers may include, but are not limited to, changes in iron metabolism and/or changes in levels of erythropoietin, haptoglobin and hepcidin levels. In some embodiments, the methods of treatment may result in a change in a patient's symptoms associated with iron overload associated with baseline transfusion therapy. Changes in iron overload symptoms may include a decrease in endocrine dysfunction caused by iron deposition in endocrine organs. Endocrine dysfunction may be evaluated by measurement of factors (levels and/or activity) such as, but not limited to, thyroid hormones, IGF-1, morning cortisol, adrenocorticotropic hormone (ACTH), HbA1C, and/or vitamin D. Determination of all the above factors, including HbA, HbF, erythropoietin, haptoglobin, hepcidin, thyroid hormones, IGF-1, cortisol, ACTH and vitamin D may be measured by standard clinical laboratory protocols.

In some embodiments, the uses and methods of treatment described herein will result in a decrease in the need for (use of) RBC transfusions and infusion of other blood products including, but not limited to, platelets, intravenous immunoglobin (IVIG), plasma and granulocytes in a subject with β-thalassemia (for example, TDT). Change in the use of RBC and other blood product infusions in a subject treated with the methods and compositions of the invention can be evaluated by keeping a log of use for the subject. The log can be used to calculate an annualized frequency and volume of packed red blood cells (PRBC) after infusion with the compositions disclosed herein, and compared to the subject's past PRBC and other blood products usage prior to treatment.

In some embodiments, the methods of treatment as described herein result in a decrease in liver disease. Liver disease and hepatomegaly are common co-morbidities of TDT due to increased red blood cell (RBC) destruction and extramedullary erythropoiesis. The accelerated rate of erythropoiesis enhances dietary iron absorption from the gut, resulting in a chronic state of iron overload analogous to that seen in hereditary hemochromatosis. Changes in iron deposition in the liver can be evaluated by MRI where iron deposition in hepatocytes and Kupfer cells can be assessed using standard methods such as the R2 based FERRISCAN® (Resonance Health) technique (see, e.g., St Pierre et al. (2013) Magn Reason Med 71(6):2215-23).

In some embodiments, the methods of treatment described herein result in a decrease in cardiac abnormalities. Cardiac abnormalities, including heart failure and fatal arrhythmias, are major complications of TDT and frequent causes of death. Life-long transfusion therapy ameliorates cardiac pathology; however, TDT patients frequently develop cardiac hemosiderosis due to myocardial iron deposition (He et al. (2008) Magn Reason Med 60(5):1082-1089). Changes in cardiac abnormalities may be evaluated by MRI, as iron deposition and overload in the myocardium can be seen in the standard myocardial T2* (T2 star) magnetic resonance technique.

In some embodiments, the methods of treatment described herein result in a decrease in osteoporosis and fractures which are a common complication of TDT (Vogiatzi et al. (2009) J Bone Miner Res 24(4543-57). Changes in bone mineral density, osteoporosis and fracture risk as a result of the methods disclosed herein can be evaluated using a standard DXA bone densitometry scan (dual energy x ray absorptiometry DXA, see e.g., Blake and Fogelman (2007) Postgrad Med J 83(982):509-517).

In some embodiments, the methods of treatment described herein result in a change (e g., reduction or increase) in baseline erythropoiesis in terms of morphology of and/type of erythroid precursor cells. TDT leads to profound erythroid hyperplasia with a high degree of immature cells and erythroid precursors of often bizarre morphologies. The methods and compositions of the invention can result in the presence of fewer immature cells and/or reduce the number of cells with non-typical morphologies. Changes in erythropoiesis can be evaluated by standard bone marrow aspiration which is a routine clinical procedure to characterize hematopoiesis.

In some embodiments, the methods of treatment described herein result in a change from baseline in the number and percent of F cells. F cells are RBCs that contain measurable amounts of HbF. Evaluation of a change in F cells as a result of the treatment methods can be measured by methods known in the art (see e.g., Wood et al. (1975) Blood 46(5):671). In certain embodiments, the number and/or percentage of F cells is increased in a subject treated as described herein, as compared to an untreated subject.

Disclosed herein are compositions comprising one or more mRNAs encoding one or more ZFNs that cleave an endogenous BCL11A sequence (e.g., an endogenous BCL11 A enhancer sequence). In certain embodiments, the one or more mRNAs comprise SB-mRENH1 mRNAs and/or SB-mRENH2 mRNAs (as shown in SEQ ID NO:15 and SEQ ID NO:16). Also disclosed are pharmaceutical compositions comprising one or more of the same or different mRNAs, including compositions comprising SB-mRENH1 and SB-mRENH2 mRNAs.

Isolated cells and isolated populations of cells comprising one or more mRNAs and/or one or more pharmaceutical compositions comprising these mRNAs are also provided. Also described are compositions comprising genetically modified cells and cells descended therefrom, including, but not limited to, progeny of the genetically modified cells. The genetically modified progeny cells may be obtained by in vitro methods (culture of the genetically modified cells) and/or in vivo following administration of the genetically modified cells to a subject. Thus, the genetically modified progeny cells may include fully or partially differentiated progeny descended from the genetically modified cells. In certain embodiments, the genetically modified cell compositions comprise genetically modified hematopoietic stem cells (also referred to as hematopoietic progenitor stem cells (HPSC) or hematopoietic stem cell/precursor cells (HSC/PC)) and/or genetically modified cells descended or produced (cultured) therefrom, including genetically modified cells in which the BCL11A sequence is cleaved and hemoglobin (e.g., HbF and/or HbA) levels in the cells are increased (e.g., 3 to 4-fold or more) as compared to cells which are not genetically modified. Some, all or none of the genetically modified cells of the cell populations and compositions of cells described herein may comprise one or more mRNAs and/or pharmaceutical compositions comprising these mRNAs. Thus, described herein are cells, cell populations and compositions comprising these cells, which cells, cell populations and compositions comprise genetically modified cells comprising the mRNAs described herein and cells descended therefrom. The cells, cell populations and compositions comprising these cells and cell populations may comprise autologous and/or allogeneic cells. Pharmaceutical compositions comprising genetically modified cells (e.g., erythroid progenitor cells such as HPSCs that exhibit increased globin expression as compared to unmodified cells) as described herein are also provided.

Methods of manufacturing (making) genetically modified isolated cells (or cell populations or compositions comprising genetically modified cells and cells descended therefrom) are also provided, including methods of making genetically modified populations of cells in which a BCL11A sequence (e.g., enhancer sequence) is genetically modified such that hemoglobin (e.g., HbF and/or HbA) levels in the genetically modified cells are increased as compared to unmodified cells (e.g., 2 or more fold). In certain embodiments, the methods comprising administering one or more mRNAs (or pharmaceutical compositions comprising the one or more mRNAs) as described herein to the cell (e.g., via transfection). The cells may be autologous and/or allogeneic and may be HSPCs. In certain embodiments, the methods further comprise culturing the genetically modified cells to produce a composition comprising a population of genetically modified cells (e.g., RPSC cells) and/or genetically modified cells descended therefrom (e.g., other erythroid progenitor cells and/or mature erythroid cells such as RBCS) exhibiting increased globin production. The compositions may comprise genetically modified cells comprising the mRNAs and/or genetically modified cells descended from such cells that no longer comprise the mRNAs but maintain the genetic modification (BCL11A-specific modifications). Pharmaceutical compositions comprising genetically modified cell populations and/or cells descended therefrom are also provided.

Thus, in some embodiments, the methods and compositions disclosed herein relate to treating a subject with cells that have been modified ex vivo. In some embodiments, the cells are isolated from the subject, modified ex vivo, and then returned to the subject. In other embodiments, the cells are isolated from healthy donors, modified ex vivo, and then used to treat the subject. In further embodiments, the cells isolated from healthy donors are further modified ex vivo to remove self-markers (e.g., HLA complexes) to avoid rejection of the cells by the subject. In some embodiments, the cells isolated are stem cells. In further embodiments, the stem cells are hematopoietic stem cell progenitor cells (e.g., CD34+ HSC/PC). In some embodiments, the CD34+ HSC/PC are mobilized in each subject by treatment with one or more doses of granulocyte colony-stimulating factor (G-CSF) In some embodiments, the dose of G-CSF used is about 10 μg/kg/day. In some embodiments, the one or more doses of G-CSF are combined with one or more doses of plerixafor. In some embodiments, the dose of plerixafor used is about 240 μg/kg/day. In further embodiments, the mobilized cells are harvested by one or more apheresis cycles.

Mobilized human CD34+ HSPCs may be collected by apheresis from healthy or beta-thalassemia subjects and purified prior to administration of (transfection with) one or more mRNAs (or pharmaceutical compositions comprising the one or more mRNAs) as described herein. In certain embodiments, the purified HSPCs are transfected with IFN mRNAs SB-mRENH1 and SBmRENH2 (SEQ ID NO:15 and SEQ ID NO:16). Transfected genetically modified CD34+ HSPCs (“ST-400”) may be cultured, harvested and/or frozen for use. After harvesting, compositions comprising genetically modified cells (at least 50%, preferably at least 70% or more, even more preferably at least 75-80% or more of the cells are genetically modified following mRNA. administration, preferably specifically modified at the BCL11A enhancer sequence as compared to other genetic loci) as described herein (“ST-400”) may include HSPCs as well as cells descended therefrom, for instance HSPC differentiated into all hematopoietic lineages, including erythroid progenitors (CFU-E and BFU-E), ganulocyte/macrophage progenitors (CFU-G/M/GM), and multi-potential progenitors (CFU-GEMM). In certain embodiments, some, none or all of the genetically modified cells of the composition (population) of cells comprise one or more of mRNAs.

In any of methods or uses described herein, the subject has a confirmed molecular genetic diagnosis ofβ-thalassemia; confirmed clinical diagnosis of β-thalassemia (e.g., TDT); is β⁰/β⁰ or non-β⁰/β⁰ and/or is between the ages of 18 and 40 years old with a clinical diagnosis β beta-thalassemia (e.g., TDT) with ≤8 documented PRBC transfusion events per year on an annualized basis in the prior two year period. In certain embodiments, the genetically modified CD34+ HSPCs are generated from cells obtained from the subject (autologous). In certain embodiments, CD34+ HSPCs are mobilized in each subject using treatment with G-CSF and plerixafor. Mobilized CD34+ HSPCs are collected from each subject one or more days (e.g., 3, 4, 5, 6, 7 or more days) after mobilization by apheresis, for example on 2 or more consecutive days until sufficient cells are collected. In certain embodiments, at least about 1×10⁴ to 1×10⁷ (e.g., 25×10⁶) CD34+ HSPCs/kg are collected. If needed, a second mobilization and apheresis cycle may be performed 1, 2, 3 or more weeks after the first cycle. In certain embodiments, a portion of collected cells are subject to genetic modification as described herein and the remainder maintained (e.g., cryopreserved) in the event a rescue treatment for the subject is indicated.

In some embodiments, the cells are removed from the subject (autologous) and treated with nucleases that target a gene involved in the regulation of fetal hemoglobin (HbF) production. hi some embodiments, the gene is a repressor of HbF production. In some embodiments, the gene is the BCL11A gene. In some embodiments, the nucleases target and cleave the erythroid-specific enhancer region of the BCL11A gene. In some embodiments, the nucleases are delivered to the cells as mRNAs. In some embodiments, the cleavage of the erythroid-specific enhancer region results in error-prone repair of the cleavage site by the cellular repair machinery such that a binding site for the erythroid transcription factor GATA1 (see Vierstra et al. (2015) Nat Methods 12(10):927-30; Canver et al. (2015) Nature 527(7577):192-7) is disrupted. In some embodiments, the nucleases target the erythroid-specific enhancer region of the BCL11A gene such that it is not expressed in hematopoietic stem cells. Enhancer regions targeted may be within or outside the coding region including but not limited to +58, +55 and/or +62 regions within intron 2 of endogenous BCL11A, numbered in accordance with the distance in kilobases from the transcription start site of BCL11A, which enhancer regions are roughly 350 (+55), 550 (+58); and 350 (+62) nucleotides in length. See, e.g., Bauer et al. (2013) Science 343:253-257; U.S. Pat. Nos. 9,963,715; 10,072,066; and U.S. Patent Publication Nos. 2015/0132269 and 2018/0362926. In some embodiments, the modified HSC/PC are evaluated prior to returning to the subject. In some embodiments, the modified cells are evaluated for the presence and type of nuclease-induced mutations in the BCL11A enhancer region. In some embodiments, the mutations can be insertions of nucleotides, deletions of nucleotides or both (“indels”). In some embodiments, the cells are evaluated for off-target cleavage by the nucleases. In some embodiments, the cells are evaluated for molecular translocations and/or karyotyping of the cellular chromosomes following nuclease cleavage. In some embodiments, the cells are evaluated for off-target transcriptional activity. In some embodiments, the cells are evaluated for endotoxin load. In some embodiments, the cells can be evaluated for one or more of the above characteristics.

In some embodiments, the modified CD34+ HSC/PC are returned to the subject at a dose such that HbF production is increased and the clinical symptoms of β-thalassemia are decreased. In some embodiments, the subject is treated with one or more myeloablative condition agents prior to infusion with the modified CD34+ HSC/PC. In some embodiments, the myoelablative agent is busulfan. In further embodiments, the busulfan is used with other agents such as cyclophosphamide.

In some embodiments, a dose of about 3×10⁶ cells/kg to about 20×10⁶ cells/kg (or any value therebetween) of the genetically modified cells is administered (e.g., via intravenous infusion) to the subject. In some embodiments, the cells are formulated in infusible cryomedia containing 10% DMSO. In some embodiments, the cells are formulated with approximately 1-2.0×10⁸ cells per bag at a concentration of approximately 1×10⁷ cells/mL. In any of the methods described herein, cells dosages may be determined as total cell dose or as a CD34+ cell dose, which can be calculated as follows: CD34+ dose=[total cell dose]×[CD34+%]. See, e.g., Table B, showing total cell dose in column 2 and CD34+% in column 3. In some embodiments, subjects receiving the modified HSPC are monitored after infusion for engraftment of the modified cells and for evaluating the heterogenicity of the modified cell population. In some embodiments, peripheral blood, bone marrow and/or different cellular populations may be individually assessed for the presence of indels in the BCL11A gene. In some embodiments, genomic DNA from cells isolated from a treated subject is isolated and the region comprising the BCL11A target sequence is amplified. In further embodiments, the percent modified cells within the cell population is determined and re-tested over time post dosing to evaluate stability of the modified cell population with the treated subject.

In some embodiments, the modification data is evaluated to create an indel profile. In further embodiment, the indel profile is monitored over time to determine the likelihood of any one particular cell type (indel profile) aberrantly overgrowing the population.

Disclosed herein are compositions and methods for treating a subject with β-thalassemia comprising cells that have been treated with two polynucleotides encoding partner halves (also referred to as a “paired ZFN” or “left and right ZFNs”) of a zinc finger nuclease. Optionally, the nuclease-encoding polynucleotides further comprise sequences encoding small peptides (including but not limited to peptide tags and nuclear localization sequences), and/or comprise mutations in one or more of the DNA binding domain regions (e.g., the backbone of a zinc finger protein) and/or one or more mutations in a FokI nuclease cleavage domain or cleavage half domain. The polynucleotides may optionally comprise an ARCA cap (U.S. Pat. No. 7,074,596). When these polynucleotide components are used individually or in any combination (e.g., peptide sequence such as FLAG, NLS, WPRE, ARCA and/or poly A signal in any combination), the methods and compositions of the invention provide surprising and unexpected increases in expression of artificial nucleases with increased efficiency (e.g., 2, 3, 4, 5, 6, 10, 20 or more fold cleavage as compared to nucleases without the sequences/modifications described herein) and/or targeting specificity. In certain embodiments, described herein is a composition comprising genetically modified cells specifically modified at the BCL11A locus by the mRNA(s) as described herein, including in which less than 10% (0 to 10% or any value therebetween), preferably less than 5% (0 to 5% or any value therebetween), even more preferably less than 1% of the cells (0 to 1% or any value therebetween) and even more preferably less than 0.5% (0 to 1% or any value therebetween) of the genetically modified cells include genetic modifications made by the mRNA(s) outside the BCL11A locus (but may include additional modifications such as inactivation of HLA markers). In further embodiments, the polynucleotides encoding the zinc finger nuclease may comprise a left ZFN known as SB63014 (see, U.S. Pat. No. 10,563,184 and U.S. Patent Publication No. 2018/0087072), encoded by a mRNA SB-mRENH1. In some embodiments, the right ZFN is SB65722 (see, U.S. Pat. No. 10,563,184 and U.S. Patent Publication No. 2018/0087072), encoded by a mRNA SB-mRENH2.

Also described herein are host cells, including isolated hematopoietic stem cells (HSPC such as CD34+), comprising the ZFNs and/or polynucleotides (e.g., mRNAs) as described herein. Cells may be isolated from healthy subjects or, alternatively, are autologous cells obtained from a subject with the condition to be treated (e.g., TDT) and purified using standard techniques. The ZFNs genetically modify the cells via insertions and/or deletions following cleavage. Subsequently, expanded (cultured) cells may no longer include the ZFNs (or polynucleotides encoding these ZFNs) but maintain the genetic modifications in culture (e.g., insertions and/or deletions within BCL11a). In certain embodiments, the genetic modifications are insertions and/or deletions (“indels”) made by NHEJ following cleavage. Genetically modified cells as described herein exhibit different ratios of globin (α-, β- and γ-globin levels) as compared to untreated (non-genetically modified) cells. In certain embodiments, the ratio of γ-globin to β-globin and of γ-globin to α-globin is increased about 2 to 5 or more-fold, including 3 to 4-fold as compared to untreated (untransfected) HSPCs. Furthermore, the genetically modified cells described herein differentiate into all hematopoietic lineages, including erythroid progenitors (CFU-E and BFU-E), granulocyte/macrophage progenitors (CFU-G/M/GM), and multi-potential progenitors (CFU-GEMM) and exhibit normal karyotypes and morphology, which is indicative of a reconstitution of hematopoiesis.

In certain aspects, ex vivo therapies for TDT are described using the genetically modified cells as described herein. In certain embodiments, the genetically modified cells are autologous cells obtained from the subject to be treated, which cells are then genetically modified as described herein and administered back to the same subject. Cells obtained from the subject may be mobilized in the subject using treatment with G-CSF and/or plefixafor. See, FIG. 5. In any of the methods described herein, any amount of cells may be mobilized, for example about 5×10⁵, about 10×10⁵, about 15×10⁵, about 20×10⁵, about 5×10⁶, about 10×10⁶, about 15×10⁶, about 20×10⁶, about 25×10⁶ CD34+ HSPCs/kg for genetic modification are mobilized in the subject. The autologous cells are genetically modified as described herein and cryopreserved (e.g., using a controlled rate freezer) according to standard techniques with each aliquot (e.g., infusion bag) having a total cell count of approximately 1.0×10⁸ to 2.0×10⁸ cells and can be stored in vapor phase liquid nitrogen (at <−150° C.) at the manufacturing facility until they are ready to be shipped to the clinical study center.

In any of the methods described herein, the subject can receive conditioning therapy prior to ex vivo therapy with genetically modified cells, for example, via intravenous (IV) administration of busulfan using an effective dose and regimen. According to standard procedures, for example, busulfan is dosed at between about 0.5 to 5 mg/kg (or any value therebetween). In certain embodiments, subjects will receive a myeloablative regimen of busulfan (about 3.2 mg/kg/day; IV via central venous catheter) for up to 4 days (total dose of about 12.8 mg/kg prior to infusion), for example on Days −6 through −3 before infusion of the modified HSPC on Day 0. IV busulfan may be dosed once daily (total of 4 doses) or every 6 hours (total of 16 doses) according to study center practices or guidelines. After the first dose, the IV busulfan dose will be adjusted based on pharmacokinetic sampling and study center practices to target an area under the curve (AUC) of 4,000-5,000 mmol*min for daily dosing or an AUC of 1,000-1,250 mmol*min for every 6 hour dosing for a total regimen target AUC of 16,000-20,000 mmol*min. IV busulfan pharmacokinetic targeting may be modified for subsequent subjects. Optionally, therapeutic drug monitoring is conducted to determine clearance of busulfan after 4 days of dosing is complete.

In certain aspects, the ex vivo therapies comprise thawing the frozen genetically modified HSPC and infusing the cells into the subject, preferably within about 15 to about 45 minutes of thawing. The volume of frozen modified HSPC administered is determined by the subject's weight. Vital signs (blood pressure, temperature, heart rate, respiratory rate and pulse oximetry) are monitored prior to infusion and afterwards. In certain embodiments, the subjects are monitored using blood tests as well as analysis of HbF levels (baseline levels of HbF fractions (A and F in g/dL) and percent HbF is determined based on the last assessment on or prior to the date of first administration of IV busulfan), endocrine function, and/or performing MRIs to assess iron load. In certain embodiments, the ex vivo therapies result in neutrophil and platelet recovery to within normal levels in the TDT subject from within about two to four weeks of infusion. Subjects may also receive PRBC transfusions 0, 1 or more times following HSPC infusion. In certain embodiments, by weeks (e.g., 2, 3, 4, 5, 6, 7 or more) after infusion with the modified HSPC, total hemoglobin levels remain stable or continue to rise in the subject.

Following infusion, the modified HSPC may be monitored in the patient to determine engraftment efficiency and/or modification heterogenicity. This can be done, for example, by determining the genetic modification (“indel”) profile. Cell samples may be purified from the peripheral blood, bone marrow aspirate or other tissue samples (preferably about 5×10⁴ to 1×10⁷ cells) and subject to genomic DNA isolation for assessment. Bone marrow aspirate or other tissue samples may be taken at various timepoints, including at between about 6-9 months.

In some embodiments, provided herein are methods of treatment that reduce, delay, and/or eliminate additional treatment procedures as compared with a subject that has not been treated with the methods and compositions as disclosed herein, for example wherein an effective amount of modified HSC/PC are administered to a subject in need thereof, wherein the subject has a reduced, delayed, and/or eliminated need for additional treatment procedures after treatment. In some embodiments, the additional treatment procedures can include, but are not limited to, a bone marrow transplant, PRBC and/or other blood component transfusions, and treatments related to iron chelation therapy.

In some embodiments, the ZFN useful in the compositions and methods disclosed herein (e.g., a ZFN in which the members of the ZFN pair (left and right) ZFNs are delivered by two separate mRNAs) include mRNAs designated SB-mRENH1 and SB-mRENH2. In some embodiments, the ZFNs in the BCL11A-specific pair are delivered (e.g., to the HSC/PC) via electroporation, for example, wherein one AAV comprises the left ZFN SB-mRENH1) and another comprises the right ZFN (e.g., SB-mRENH2).

Thus, described herein are methods for altering hemoglobin expression for the treatment and/or prevention of β-thalassemia (for example TDT). In certain embodiments a ZFN pair comprising first and second (left and right) ZFNs, namely a 6-finger ZFN comprising a ZIP designated 63014 comprising the recognition helix regions as shown in Table 1 (e.g., encoded by mRNA SB-mRENH1) and a 5-finger ZFN comprising a ZFP designated 65722 comprising the recognition helix regions as shown in Table 1 (e.g., encoded by mRNA SB-mRENH2) is used for altering hemoglobin levels in an isolated cell or cell of a subject, including for the treatment of TDT. The ZFN pair binds to a 33-base pair (combined) target site in the erythroid-specific enhancer of the human BCL11A gene at location chr2:60,495,250-60,495,290 in the GRCh38/hg38 assembly of the human genome. In certain embodiments, one mRNA encodes both ZFNs of the pair. Alternatively, separate mRNAs, each encoding one ZFN of the pair are employed. In certain embodiments, the mRNA sequences are shown in Example 1 (SEQ ID NO:15 and SEQ ID NO:16).

Optionally, the nuclease-encoding polynucleotides further comprise sequences encoding small peptides (including but not limited to peptide tags and nuclear localization sequences), and/or comprise mutations in one or more of the DNA binding domain regions (e.g., the backbone of a zinc finger protein or TALE) and/or one or more mutations in a FokI nuclease cleavage domain or cleavage half domain. When these polynucleotide components are used individually or in any combination (e.g., peptide sequence such as FLAG, NLS, WPRE and/or poly A signal in any combination), the methods and compositions of the invention provide surprising and unexpected increases in expression of artificial nucleases with increased efficiency (e.g., 2, 3, 4, 5, 6, 10, 20 or more fold cleavage as compared to nucleases without the sequences/modifications described herein) and/or targeting specificity. Thus, according to certain embodiments, the cells (populations of cells and compositions comprising these cells and populations of cells) described herein are specifically genetically modified by the mRNA(s) at the BCL11A locus, including genetically modified cell populations (and compositions comprising these cells) in which less than 10% (0 to 10% of any value therebetween), preferably less than 5% (0 to 5% or any value therebetween), even more preferably less than 1% of the cells to 1% or any value therebetween) and even more preferably less than 0.5% (0 to 1% or any value therebetween) of the genetically modified cells include genetic modifications made by the mRNA(s) outside the BCL11A locus (but may include additional modifications such as inactivation of HLA markers). In some embodiments, the nuclease is encoded by an mRNA and the mRNA optionally comprises elements for increasing transcriptional and translational efficiency.

The methods and compositions of the invention can also include mutations to one or more amino acids within the DNA binding domain outside the residues that recognize the nucleotides of the target sequence (e.g., one or more mutations to the ‘ZFP backbone’ (outside the DNA recognition helix region)) that can interact non-specifically with phosphates on the DNA backbone. Thus, in some embodiments, the methods and compositions disclosed herein includes mutations of cationic amino acid residues in the ZFP backbone that are not required for nucleotide target specificity. In some embodiments, these mutations in the ZFP backbone comprise mutating a cationic amino acid residue to a neutral or anionic amino acid residue. In some embodiments, these mutations in the ZFP backbone comprise mutating a polar amino acid residue to a neutral or non-polar amino acid residue. In some embodiments, mutations at made at position (−5), (−9) and/or position (−14) relative to the DNA binding helix. In some embodiments, a zinc finger may comprise one or more mutations at (−5), (−9) and/or (−14). In some embodiments, one or more zinc fingers in a multi-finger zinc finger protein may comprise mutations in (−5), (−9) and/or (−14). In some embodiments, the amino acids at (−5), (−9) and/or (−14) (e.g., an arginine (R) or lysine (K)) are mutated to an alanine (A), leucine (L), Ser (S), Asp (N), Glu (E), Tyr (Y) and/or glutamine (Q). See, e.g., U.S. Patent Publication No. 2018/0087072.

In some aspects, the methods and compositions of the invention include the use of sequences encoding exogenous peptide sequences fused to eukaryotic transgene sequences. In some embodiments, exogenous peptides are fused to protein sequences post-translationally, and in other embodiments, the sequences encoding the exogenous peptides are linked in frame (3′ and/or 5′) to sequences encoding the artificial nuclease (e.g., a fusion protein). In preferred embodiments, a sequence encoding 3 FLAG sequences (3× FLAG peptide) is used (see, U.S. Pat. No. 6,379,903), wherein the amino acid sequence is N-term. DYKDHDG-DYKDHDI-DYKDDDDK (SEQ ID NO:1). Inclusion of one or more of such peptide sequences (e.g., 3× FLAG) can increase nuclease (cleavage) activity by 2, 3, 4, 5, 6, 7, 8, 9, 10, 11 or more-fold as compared to nucleases without the peptide sequences.

In some aspects, the mRNA encoding an artificial nuclease comprises a nuclear localization peptide sequence (NLS). In some embodiments, the NLS comprises the sequence PKKKRKV (SEQ ID NO:2) from the SV40 virus large T gene (see, Kalderon et al. (1984) Nature 311(5981):33-8). Inclusion of one or more of NLS sequences as described herein can increase nuclease (cleavage) activity by 2, 3, 4, 5, 6, 7, 8, 9, 10, 11 or more-fold) as compared to nucleases without the peptide sequences.

In some embodiments, the methods and compositions disclosed herein comprise dosing of a composition of the invention (for example, the modified HSC/PC), for example, via a peripheral vein catheter. In some embodiments, the composition is administered to the subject which is then followed by administration of normal saline (NS) or phosphate buffered saline (PBS). In some embodiments, the subject receives a total dose of modified cells of between about 3.0×10⁶ cells/kg and about 20×10⁶ cells/kg (or any value therebetween). Any dose in the range of about 3.0×10⁶ to about 20×10⁶ cells/kg may be used.

In some embodiments, the subject has delayed, reduced or eliminated need, for example, for additional therapeutic procedures after receiving a total dose of between about 10×10⁶ to about 20×10⁶ cells/kg.

In another aspect, disclosed herein is a method of reducing, delaying or eliminating the thalassemia-related disease biomarkers following treatment with the methods and compositions in a subject with β-thalassemia as compared with the subject prior to treatment with the methods and compositions of the invention. Determination of thalassemia-related biomarkers, including HbA, HbF, erythropoietin, haptoglobin, hepcidin, thyroid hormones, IGF-1, cortisol, ACTH and vitamin D may be measured by standard clinical laboratory protocols, the method comprising, for example, administering to the subject an effective amount of modified HSC/PC wherein the subject has reduced, delayed or eliminated thalassemia-related disease biomarkers after treatment. In some embodiments, levels of HbF increase by about 5%, 10%, 20%, 30%, 40%, 50%, 60%, 70%, 80%, 90%, 100%, 150%, 200%, 300%, 400% or more (or any value therebetween) following treatment by the methods disclosed herein.

In another aspect, disclosed herein is a method of reducing, delaying or eliminating the use of PRBC transfusions and infusion of other blood products including, but not limited to, platelets, intravenous immunoglobin (IVIG), plasma and granulocytes following treatment with the methods and compositions in a subject with β-thalassemia as compared with a subject that has not been treated with the methods and compositions of the invention. In some embodiments, the use of PRBC and/or other blood product is decreased by about 10%, 20%, 30%, 40%, 50%, 60%, 70%, 80%, 90%, 100% or any value therebetween in a subject treated with the methods disclosed herein as compared to the subject prior to receiving treatment. In some embodiments, the use of PRBC and/or other blood product infusions is eliminated.

In another aspect, disclosed herein is a method of reducing, delaying or eliminating the symptoms associated with iron overload in a subject with β-thalassemia. In some embodiments, markers of endocrine dysfunction as a result of iron deposition in endocrine organs (for example, thyroid markers, IGF-1, morning cortisol, HbA1C and Vitamin D) become normalized in a subject after treatment with the methods and compositions of the invention as compared to the marker levels prior to treatment. In some embodiments, iron overload in the liver and heart is decreased in a subject following treatment with the methods and compositions disclosed herein as compared with the subject prior to treatment. Iron overload can be evaluated by standard MRI procedures. In some embodiments, iron over load in the liver and/or heart detected by MRI is decreased by about 5%, 10%, 20%, 30%, 40%, 50%, 60%, 70%, 80%, 90%, 100% or any value therebetween in a subject treated with the methods disclosed herein as compared to the subject prior to receiving treatment.

In another aspect, disclosed herein is a method of reducing, delaying or eliminating the symptoms associated with osteoporosis and/or bone fractures in a subject with β-thalassemia. In some embodiments, bone density is increased in subjects treated with the methods and compositions disclosed herein in comparison with the subjects prior to treatment. In some embodiments, osteoporosis and bone fractures are reduced or eliminated in a subject treated with the methods and compositions disclosed herein in comparison with the subject prior to treatment. In some embodiments, osteoporosis and/or bone fractures are ameliorated by about 5%, 10%, 20%, 30%, 40%, 50%, 60%, 70%, 80%, 90%, 100% or any value therebetween in a subject treated with the methods disclosed herein as compared to the subject prior to receiving treatment.

In another aspect, disclosed herein is a method of reducing, delaying or eliminating erythroid hyperplasia in a subject with TDT, the level of immature cells and erythroid precursors in the hone marrow in a subject following treatment with the methods and compositions disclosed herein as compared to the subject prior to treatment.

In another aspect, provided herein is an article of manufacture comprising a package (for example, a bag) comprising compositions comprising genetically modified autologous HSC/PC as described herein. The article of manufacture (e.g., bag) may be formulated for frozen storage, for example in CryoStor® CS-10 cryomedia (SigmaAldrich) containing 10% DMSO. Each bag can contain any concentration of cells. In certain embodiments, each bag contains approximately 1.0-2.0×10⁸ cells per bag at a concentration of approximately 1×10⁷ cells/mL.

In a further aspect, described herein are methods of monitoring the modification profile (e.g., number and/or types of insertions and/or deletions generated following cleavage, typically by NHEJ of the cleaved sequence) a population of genetically modified cells as described herein. The monitoring may be conducted before and/or after administration to the subject to determine if one type of modification (clone) predominates in the population, as such jackpotting may result in unwanted proliferation of a particular clonal population. In certain embodiments, the population of genetically modified cells is monitored for the type of modification (insertions and/or deletions, also referred to as “indel/profile”) using standard techniques such as sequencing or the like. In certain embodiments, the population of cells is assayed prior to administration to determine a baseline of the pattern of modifications (indel profile) and subsequently monitoring after infusion to determine that the indel profile of the engrafted cells is being maintained, such there is not aberrant outgrowth of one clonal population of cells. The monitoring may be conducted over time (multiple times) before and/or after infusion. Thus, the methods described herein may further comprise monitoring the population of genetically modified cells before and/or after infusion to determine the indel profile is remaining the same over time.

From the description herein, it will be appreciated that that the present disclosure encompasses multiple embodiments which include, but are not limited to, the following:

Genetically modified cells comprising red blood cell (RBC) precursor cells comprising SB-mRENH1 mRNAs and SB-mRENH2 mRNAs, which mRNAs encode a ZFN pair; and a genomic modification made following cleavage by the ZFN pair, wherein the modification is within an endogenous BCL11A enhancer sequence, such that the BCL11A gene is inactivated in the cell. Also included are cells descended therefrom.

An ex vivo method of treating a beta-thalassemia (β-thalassemia) in a subject in need thereof, the method comprising: administering a composition according to any of the embodiments described herein to the subject such that fetal hemoglobin (HbF) production in the subject is increased and one or more clinical symptoms of β-thalassemia are decreased, ameliorated, or eliminated.

An ex vivo method according to any of the preceding embodiments described herein, wherein the beta-thalassemia is transfusion-dependent β-thalassemia.

An ex vivo method according to any of the preceding embodiments described herein, wherein a change from baseline of clinical laboratory hemoglobin factions in grams/dL plasma and/or percent HbF of total hemoglobin (Hb) is achieved in the subject.

An ex vivo method according to any of the preceding embodiments described herein, wherein the hemoglobin factor is adult hemoglobin (HbA) and/or fetal hemoglobin (HbF).

An ex vivo method of according to any of the preceding embodiments described herein, wherein the subject is β⁰/β⁰ or β⁰/β⁺.

An ex vivo method according to any of the preceding embodiments described herein, wherein levels of thalassemia-related disease biomarkers are altered following treatment.

An ex vivo method according to any of the preceding embodiments described herein, wherein the biomarkers are changes in iron metabolism; and/or changes in levels of erythropoietin, haptoglobin and/or hepcidin.

An ex vivo method according to any of the preceding embodiments described herein, wherein the clinical symptoms associated with iron overload or associated with baseline transfusion therapy are ameliorated or eliminated.

An ex vivo method according to any of the preceding embodiments described herein, wherein a decrease in endocrine dysfunction in the subject is assayed by determining levels and/or activity of thyroid hormones, IGF-1₉ morning cortisol, adrenocorticotropic hormone (ACTH), HbA1C, and/or vitamin D levels.

An ex vivo method according to any of the preceding embodiments described herein, wherein the need for RBC transfusions and infusion platelet transfusion, intravenous immunoglobin (IVIG) transfusion, plasma transfusion and/or granulocyte transfusion in the subject is(are) reduced or eliminated.

An ex vivo method according to any of the preceding embodiments described herein, wherein the clinical symptom reduced or eliminated in the subject is liver disease.

An ex vivo method according to any of the preceding embodiments described herein, wherein the clinical symptoms reduced or eliminated in the subject are cardiac abnormalities.

An ex vivo method according to any of the preceding embodiments described herein, wherein the clinical symptoms reduced or eliminated in the subject is/are osteoporosis and/or fractures.

An ex vivo method according to any of the preceding embodiments described herein, wherein baseline erythropoiesis is changed in the subject following administration of the composition.

An ex vivo method according to any of the preceding embodiments described herein, wherein hyperplasia is reduced or eliminated in the subject following administration of the composition.

An ex vivo method according to any of the preceding embodiments described herein, wherein the number of immature and/or cells with non-typical morphologies is/are reduced in the subject.

An ex vivo method according to any of the preceding embodiments described herein, wherein the number and percent of F cells in the subject is modified following administration of the composition.

An ex vivo method according to any of the preceding embodiments described herein, wherein the genetically modified cells are autologous or allogeneic.

An ex vivo method according to any of the preceding embodiments described herein, wherein the BCL11A-genetically modified cells further comprise one or more additional genetical modifications.

An ex vivo method according to any of the preceding embodiments described herein, wherein the genetically modified cells are allogeneic cells and the one or more additional genetic modifications comprise inactivation of one or more self-markers or antigens.

An ex vivo method according to any of the preceding embodiments described herein, wherein the genetically modified cells are hematopoietic stem cells isolated from the subject.

An ex vivo method according to any of the preceding embodiments described herein, wherein the hematopoietic stem cells are CD34+ hematopoietic stem or precursor cells (HSC/PC) and the CD34+ HSC/PC are mobilized in each subject by treatment with one or more doses of G-CSF and/or one or more doses of plerixafor prior to isolation.

An ex vivo method according to any of the preceding embodiments described herein, wherein at least 25×10⁶ CD34+ HSPCs/kg are mobilized in the subject and the mobilized cells are harvested by one or more apheresis cycles.

An ex vivo method according to any of the preceding embodiments described herein, further comprising, prior to administering the composition comprising the genetically modified cells to the subject and evaluating the cells of the composition for insertions and/or deletions within BCL11A.

An ex vivo method according to any of the preceding embodiments described herein, further comprising administering with one or more myeloablative condition agents one or more times to the subject prior to administration of the composition comprising the genetically modified cells.

An ex vivo method according to any of the preceding embodiments described herein, wherein the myeloablative agent comprises busulfan and further wherein: intravenous (IV) administration of the busulfan is between 0.5 to 5 mg/kg for one or more times; IV administration of the busulfan is 3.2 mg/kg/day; IV via central venous catheter for 4 days total dose of 12.8 mg/kg prior to infusion on Days −6 through −3 before infusion of the composition comprising the genetically modified cells on Day 0; or IV administration of the busulfan is once daily or every 6 hours.

An ex vivo method according to any of the preceding embodiments described herein, wherein the dose of genetically modified cells administered to the subject is between 3×10⁶ cells/kg and 20×10⁶ cells/kg.

An ex vivo method according to any of the preceding embodiments described herein, wherein the genetically modified cells administered to the subject are formulated with approximately 1×2.0×10⁸ cells per bag at a concentration of approximately1×10⁷ cells/mL.

An ex vivo method according to any of the preceding embodiments described herein, wherein the genetically modified cells are cryopreserved prior to administration and are administered to the subject within about 15 minutes of thawing.

An ex vivo method according to any of the preceding embodiments described herein, further comprising monitoring the subject's vital signs prior to, during and/or after administration of the genetically modified cells.

An ex vivo method according to any of the preceding embodiments described herein, further comprising assessing hemoglobin, neutrophil and/or platelet levels in the subject prior to administration of the genetically modified cells to determine baseline levels of hemoglobin in the subject.

An ex vivo method according to any of the preceding embodiments described herein, wherein hemoglobin, neutrophil and/or platelet levels in the subject after administration of the genetically modified cells increase or remain stable as compared to baseline levels fir weeks or months after administration.

An ex vivo method according to any of the preceding embodiments described herein, wherein the subject receives one or more packed red blood cell (PRBC)transfusions prior to and/or after administration of the genetically modified cells.

An ex vivo method according to any of the preceding embodiments described herein, wherein the need for additionally therapies such as a bone marrow transplant, blood component and/or iron chelation therapy PRBC transfusions in the subject are reduced or eliminated.

An ex vivo method according to any of the preceding embodiments described herein, wherein the need for additional therapies is reduced or eliminated within 1-20 days of administration of the genetically modified cells.

An ex vivo method according to any of the preceding embodiments described herein, wherein the subject is monitored over time post administration to determine the indel profile of cells isolated from peripheral blood samples, bone marrow aspirates or other tissue sources in comparison with the indel profile of the infused cells to monitor stability of the graft in the subject.

An ex vivo method according to any of the preceding embodiments described herein, wherein the indel profile of the cells is monitored prior to administration to the subject.

An article of manufacture comprising a package comprising a composition according to claim 2 formulated in CryoStor® CS-10 cryomedia.

The article of manufacture according to any of the preceding embodiments described herein, wherein each bag contains approximately 1.0-2.0×10⁸ cells per bag at a concentration of approximately 1×10⁷ cells/mL.

These and other aspects will be readily apparent to the skilled artisan in light of disclosure as a whole.

### BRIEF DESCRIPTION OF THE DRAWINGS

FIG. 1 is an illustration (adapted from Hardison & Blobel (2013) Science 342(6155):206-7) of effects of low, elevated and high fetal hemoglobin levels on subjects comprising adult hemoglobin mutations (for example sickle cell disease or β-thalassemia). Shown on the far left (“low fetal hemoglobin:”) is a subject with a mutation in adult hemoglobin and wild-type ESE BCL11A, in this case the subject has normal (low) levels of fetal hemoglobin, resulting in disease symptoms in the subject. In the middle (“elevated fetal hemoglobin”), the subject has the adult hemoglobin mutation, but also has mutations in their BCL11A gene such that BCL11A expression is decreased but not eliminated, which results in elevated fetal globin levels. The subject experiences some disease amelioration due to the fetal globin “replacing” some adult globin functioning. In the far right (“high fetal hemoglobin), the subject has the adult globin mutation but has a deletion in the BCL11A enhancer, such that the subject exhibits full expression of fetal globin. This subject will experience even greater in symptom improvement by virtue of full BCL11A inactivation.

FIG. 2 depicts fetal (also referred to as gamma globin or γ globin) levels in CD34+ HSC/PC harvested from healthy volunteers (PB-MR-003 and PB-MR-004) and modified by SB-mRENH1 and SB-mRENH2. Ratios of γ-globin (sum of the Aγ-globin and Gγ-globin peaks) to α-globin and γ-globin to β-like-globin (sum of the Aγ, Gγ, β and δ-globin peaks) as determined by UPLC analysis of protein samples from Day 21 of the erythroid differentiation of the modified HSPC are depicted under the indicated conditions. 48 hours after electroporation, the cells were harvested and frozen. Cells were thawed and used to study in vitro erythropoiesis and globin production. As shown, the ratio of γ-globin to β-globin and of γ-globin to α-globin was increased approximate 3- to 4-fold in the erythroid progeny of the treated HSC/PC compared to the untransfected cells (the protein data was also supported by measurement of γ-globin mRNA levels). In each group, the bar on the left represents the ratio of γ-globin/α-globin and the bar on the right represents the ratio of γ-globin/total β-like-globin.

FIG. 3A through FIG. 3C depict graphs showing the frequency and time course of double strand breaks in modified HSPC. FIG. 3A shows a time course of number of 53BP1 foci/cell over 7 days post-transfection (“dpt”) (Mean±SD 53BP1+foci/cell). FIG. 3B and FIG. 3C show the percent of total cells with various numbers (1 to 5+) of 53BP1 foci/cell on Day 1 (FIG. 3B) and Day 7 (FIG. 3C) post-transfection. * P<0.05 vs. control,

FIG. 4 is an illustration of the probe sets used to detect chromosomal translocations. The top panel depicts chromosome segments encompassing the BCL11A enhancer on-target site (solid) and an off-target site (hatched). The bottom panel sketches positive control reagents (gBlocks) for detection of the corresponding translocation products. Also shown are the approximate primer and probe locations used in the TaqMan assay. The checkered segment within each gBlock is a unique sequence inserted into each control reagent to distinguish it from a true translocation product and allow for monitoring of potential cross-contamination. Product 1 gBlocks were probed in the BCL11A region of the fragment. Product 2 gBlocks were probed in the off-target region of the fragment.

FIG. 5 is a schematic depicting a treatment protocol using genetically modified HSPC (also referred to as “ST-400”). “G-CSF” refers to granulocyte colony-stimulating factor; “HSPC” refers to hematopoietic stem progenitor cells; “IV” refers to, intravenous; “RBC” refers to red blood cells; and “ZFN” refers to zinc finger nuclease.

FIG. 6A and FIG. 6B are graphs depicting total hemoglobin and fetal hemoglobin in a patient treated with modified HSPC (“ST-400”) as described herein (see, e.g., FIG. 5). FIG. 6A is a shows hemoglobin F levels (% of hemoglobin) at the indicated study day. FIG. 6B shows hemoglobin levels (g/dL) on the indicated study day. Arrows show when the patient received a transfusion of PRBC. The modified HSPC were administered on day 0. The data demonstrates that the patient had an increase of fetal hemoglobin to nearly 31% of the total hemoglobin 50 days after infusion. The data also demonstrate that although the patient typically received PRBC every two weeks for the two years prior to treatment, the patient did not require any PRBC between day 10 and day 50 following ST-400 infusion.

FIG. 7A through FIG. 7C depicts the 10 most frequent indels (insertions and/or deletions) detected by next-generation sequencing of nucleated blood cells (bone marrow aspirates, circulating leukocytes, or peripheral blood mononuclear cells, as available) are shown per patient at each timepoint. FIG. 7A shows Patient 1; FIG. 7B shows Patient 2; FIG. 7C shows Patient 3. No emerging dominance worrisome for hematopoietic clonality has been observed over time. Indel naming convention: “I” refers to insertion; “D” refers to deletion; the first number refers to the start of indel from reference base pair (“*” refers to nucleotides flanking indel and could align to either side of the indel); and the number following colon refers to the number of base pairs inserted or deleted. As noted, day 56 data not available for Patient 2 (FIG. 7B).

FIG. 8 depicts HbF levels in patients 1, 2 and 3 at the indicated times post treatment with ST-400. The genotype causative of beta thalassemia for each patient is also shown in each graph.

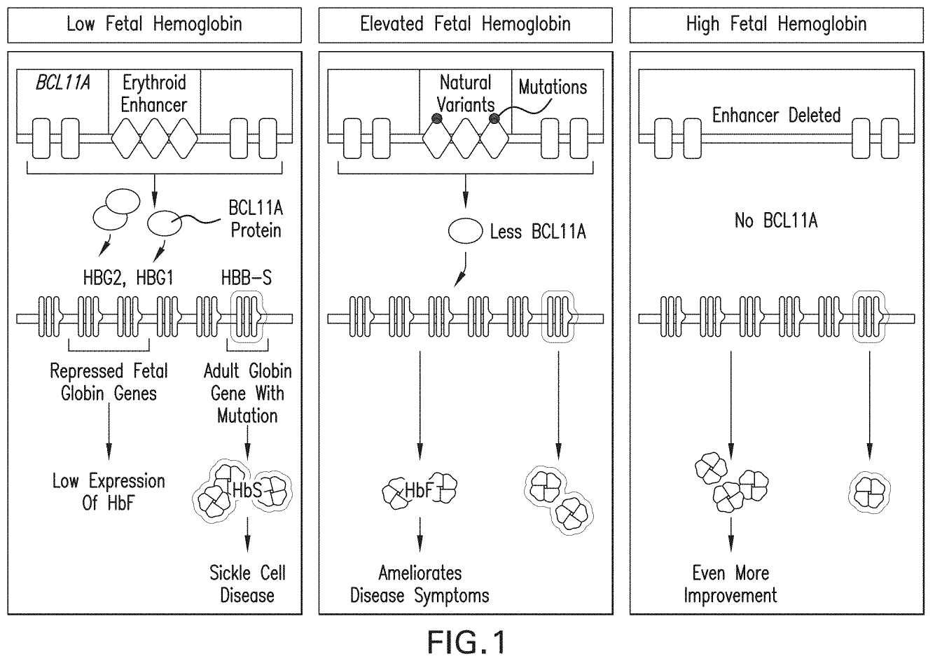

### DETAILED DESCRIPTION

Disclosed herein are compositions and methods for genome engineering for the modulation of BCL11A, gamma globin, and combinations of BLC11A and gamma globin expression and for the treatment, prevention, or treatment and prevention of hemoglobinopathies. In particular, via targeting with nucleases comprising the ZFPs having the recognition helix regions as shown in a single row of Table 1, disruption of an enhancer of BCL11A is efficiently achieved in HSC/PC and results in a change in relative gamma globin expression during subsequent erythropoiesis. This modulation of BCL11A and gamma globin expression is particularly useful for treatment of hemoglobinopathies (e.g., beta thalassemias such as TDT, sickle cell disease) wherein there is insufficient beta globin expression or expression of a mutated form of beta-globin. Using the methods and compositions described herein, the complications and disease related sequelae caused by the aberrant beta globin can be overcome by alteration of the expression of gamma globin in erythrocyte precursor cells. In particular, the compositions and methods described herein overcome the issues associated with allogeneic hematopoietic stem cell transplantation (HSCT). These issues include being limited by donor availability and the risks of graft failure and graft-vs-host disease following allogenic transplant.

High-precision gene editing of the GATA-binding region in the intronic erythroid-specific enhancer of BCL11A in hematopoietic stem or progenitor cells as described herein results in persistently high expression of fetal hemoglobin (HbF) without adversely affecting normal multi-lineage hematopoiesis. As such, the genetically modified cells can be used for ex vivo treatment of hemoglobinopathies such as TDT. Fetal hemoglobin (HbF) is the major hemoglobin present during gestation until birth. HbF is generated by combining the protein product of one of two β-like globin genes, Gγ-globin and Aγ-globin, known collectively as γ-globin, with α-globin protein as tetramers (α2γ2). HbF levels decline progressively after birth as γ-globin protein production decreases, and around 6-12 months of age is largely replaced by adult hemoglobin, which consists of a tetramer of β-globin and α-globin proteins (α2β2). Concomitant with this decline in HbF levels, the symptoms of TDT frequently become clinically apparent in infants. HbF normally only plays a minor role in normal adult physiology. However, published studies have demonstrated that congenital, acquired, and drug-induced increases in HbF are associated with reduced morbidity and improved clinical outcomes in patients with TDT. For example, large unbiased genetic studies have identified associations between TDT disease severity and quantitative trait loci such as BCL11A that is associated with increased levels of HbF (Thein et al. (2009) Hum Mol Genet 8(R2):R216-23), wherein the level of HbF is often proportional to the degree of attenuation of TDT symptomology (MusaIlam et al. (2012) Blood 119(2):364-7). Additionally, there are case reports of failed allogeneic HSCTs in TDT patients with graft rejection that serendipitously resulted in persistent high HbF levels, after which the patients were reported to be transfusion-independent (Ferster et al. (1995) Br. J Haematol 90(4):804-8; Paciaroni & Lucarelli (2012) Blood 119(4):1091-2). HbF production is increased by hydroxyurea (Walker et al. (2011) Blood 118(20):5664-70). However, hydroxyurea has been only variably effective in β-thalassemia, with greater efficacy in β-thalassemia intermedia than TDT (Charache et al. (1995) N Engl J Med 332(20):1317-22; Ansari et al. (2011) J Pediatr Hematol Oncol 33(5):339-43; Singer et al. (2008) Am J Hematol 83(11): 842-5). Furthermore, the effects of hydroxyurea are palliative, and its use requires regular monitoring for cytopenias and other toxicities.

BCL11A is a transcription factor that plays many roles in development and hematopoiesis. Genome-wide association and functional follow-up studies in cell and animal models have shown that BCL11A is an important silencer of HbF expression. In a seminal study, disruption of BCL11A by erythroid-specific conditional knockout in a transgenic humanized mouse model of sickle cell disease (SCD) lead to failure of hemoglobin switching, maintenance of high-levels of HbF, and significant improvements in the hematologic and pathologic characteristics associated with SCD (Xu et al. (2011) Science 334(6058):993-6). Thus, inhibition of BCL11 A appears to be a potentially effective strategy for treating β-globin disorders such as TDT and SCD in humans. However, targeting the BCL11 A gene for therapeutic approaches poses challenges due to the crucial role of BCL11A in development and hematopoiesis (Brendel et al. (2016) J Clin Invest 126(10:3868-3878). An alternative strategy targets an erythroid-specific enhancer (ESE) element that is located in the second intron of the BCL11A and that is required for BCL11A expression in erythroid cells but not in other lineages. The enhancer element was found to contain a common genetic variation associated with higher HbF levels (Bauer et al. (2013) Science 342(6155):253-7). It is therefore hypothesized that modification of this erythroid-specific enhancer of the BCL11A gene could boost endogenous HbF levels in erythroid cells without deleterious effects on global BCL11A function (Hardison & Blobel (2013) Science 342(6155):2067).

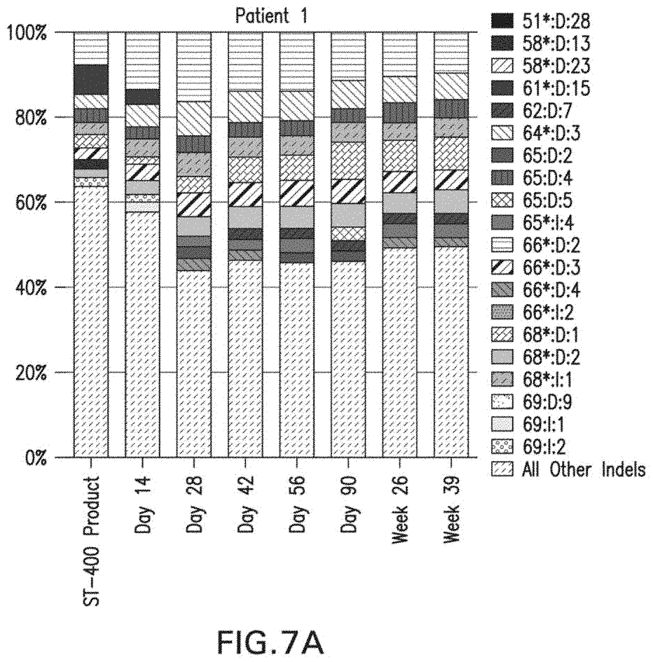

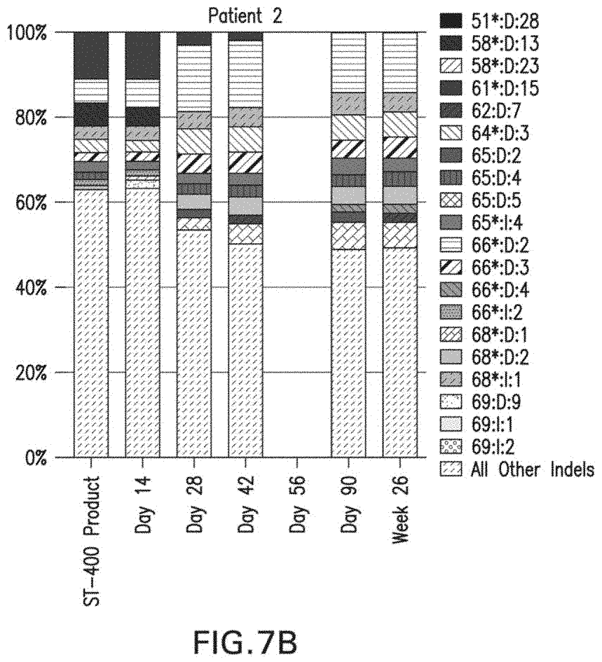

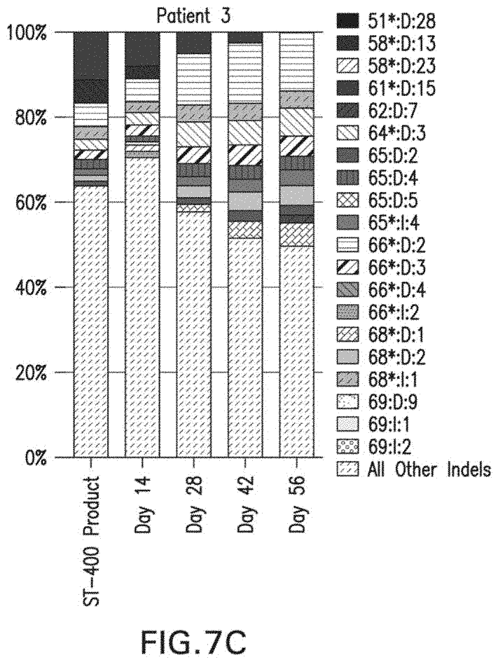

Safety of a subject following treatment with modified HSPC is of utmost concern. Thus, in any of the methods described herein, the modified HSPCs may be monitored following infusion to assess whether the modified cells are maintained in the subject over time. In addition, NHEJ following nuclease cleavage results in a population of cells that includes a variety of different insertions and/or deletions, also referred to as the indel profile. Insertions and/or deletions (indels) may be of any length and in any combination of insertions and deletions, including, but not limited to, from 0 to 10 kb nucleotides deleted; from 0 to 10 kb nucleotides inserted; from 0 to 10 kb nucleotides deleted with from 1 to 10 kb nucleotides inserted; and/or from 1 to 10 kb nucleotides deleted with from 0 to 10 kb nucleotides inserted. Indel profiles can vary widely as between patients. For instance, as shown in FIG. 7A through FIG. 7C for patients 1, 2 and 3, indels profiles for the 10 most common indels are shown for each patient, where “I” refers to insertion; “D” refers to deletion; the first number refers to the start of indel from reference base pair (“*” refers to nucleotides flanking indel and could align to either side of the indel); and the number following colon refers to the number of base pairs inserted or deleted. As shown, the most common indels varied from 1 to 28 nucleotides and started between approximately 50 and 70 (on either side) of the reference base pair. Furthermore, in all patients, “all other indels” made up over 40% of the indels evaluated. Additionally, as shown, indel profiles can change over time.

Also described herein are methods of monitoring the genetically modified HSPCs to determine their indel profile. In certain embodiments, an indel profile of the ex vivo genetically modified cells is determined before infusion and monitored over time following administration to the subject. Such monitoring assures that the pattern of distribution of indels in the engrafted cells is being maintained, and that there is not aberrant outgrowth of one clonal population of cells, a phenomenon also known as jackpotting, in which one clonal population grows faster than the rest (see, e.g., Heddle (1999) Mutagenesis 14(3):257-260), which might lead to unwanted overgrowth of a cell type derived from that modified HSPC with respect to the normal cellular homeostasis of the HSPC within the body. Monitoring of the indel profile may be conducted using any standard techniques, for example by sequencing or other method.

Thus, provided herein are genetically modified cells (e.g., red blood cell (RBC) precursor cell such as a CD34+ hematopoietic stem cell or erythroid precursor cell) comprising (i) SB-mRENH1 mRNAs and SB-mRENH2 mRNAs (as shown in SEQ ID NO:15 and SEQ ID NO:16), which mRNAs encode a ZFN pair; and (ii) a genomic modification made following cleavage bye the ZFN pair, wherein the modification is within an endogenous BCL11A enhancer sequence, such that the BCL11A gene is inactivated in the cell. Also provided are cell populations comprising these genetically modified cells; genetically modified cells descended from therefrom; cell populations comprising the genetically modified cells and cells descended therefrom; and compositions comprising the genetically modified cells and/or cells descended therefrom. The cells, cell populations, and compositions described herein may be autologous (from the subject) and/or allogeneic cells. Furthermore, the genetically modified cells may include one or more additional genetic modifications, including but not limited to cells in which one or more self-markers or antigens are inactivated (knocked-out).

Ex vivo cell therapies using these cell populations and/or compositions are also provided, for example ex vivo methods of treating a subject with beta-thalassemia (β-thalassemia) by administering a composition comprising genetically modified cells (and/or cells descended therefrom) as described herein to the subject such that fetal hemoglobin (HbF) production in the subject (e.g., β⁰/β⁰ or β⁰/β⁺) is increased and one or more clinical symptoms of β-thalassemia (e.g., transfusion-dependent β-thalassemia) are decreased, ameliorated or eliminated. In certain embodiments, a change from baseline of clinical laboratory hemoglobin fractions (adult or fetal hemoglobin) in grams/dL plasma and/or percent HbF of total hemoglobin (Hb) is achieved in the subject. In other embodiments, levels of thalassemia-related disease biomarkers (e.g., changes in iron metabolism; and/or changes in levels of erythropoietin, haptoglobin and/or hepcidin) are altered following treatment (administration of the genetically modified cells). Clinical symptoms that may be decreased, ameliorated or eliminated include but are not limited to: clinical symptoms associated with iron overload or associated with baseline transfusion therapy (e.g., a decrease in endocrine dysfunction in the subject assayed by determining levels and/or activity of thyroid hormones, IGF-1, morning cortisol, adrenocorticotropic hormone (ACTH), HbA1C, and/or vitamin D levels); the need for RBC transfusions and infusion platelet transfusion, intravenous immunoglobin (IVIG) transfusion, plasma transfusion, and/or granulocyte transfusion; liver disease; cardiac abnormalities; osteoporosis; and/or fractures. Ex vivo methods as described herein may also result in a change in baseline erythropoiesis in the subject following administration of the composition, including but not limited to, reduction or elimination of hyperplasia; reduction in the number of immature and/or cells with non-typical morphologies; and/or a change (modification) in the number and percent of F cells in the subject.

In any of the methods described herein the genetically modified cells are hematopoietic stem cells (e.g., CD34+ HSC/PC) isolated from the subject, optionally in which the CD34+ HSC/PCs are mobilized (e.g., at least 25×10⁶ CD34+ HSPCs/kg) in each subject by treatment with one or more doses of G-CSF and/or one or more doses of plerixafor prior to isolation and the mobilized cells are harvested by one or more apheresis cycles. Furthermore, the composition comprising the genetically modified cells may be evaluated for insertions and/or deletions within BCL11A (on-target modifications) and/or other non-BCL11A region (off-target modifications). Prior to administration of the composition comprising the genetically modified cells, the subject may be treated with (administered) one or more myeloablative condition agents one or more times, for example, busulfan administered: intravenously (IV) at between 0.5 to 5 mg/kg for one or more times; IV at 3.2 mg/kg/day; IV via central venous catheter for 4 days total dose of 12.8 mg/kg prior to infusion on Days −6 through −3 before infusion of the composition comprising the genetically modified cells on Day 0; or IV once daily or every 6 hours. Any dose of genetically modified cells can be used, for example, between 3×10⁶ cells/kg and 20×10⁶ cells/kg (e.g., where the cells are formulated with approximately 1.0-2.0×10⁸ cells per bag at a concentration of approximately 1×10⁷ cells/mL). The genetically modified cells may be cryopreserved prior to administration and may be at any time after thawing, including but not limited to within about 15 minutes to about 45 minutes of thawing. The methods may further comprise monitoring the subject's vital signs prior to, during and/or after administration of the genetically modified cells; and/or assessing hemoglobin, neutrophil and/or platelet levels in the subject prior to administration of the genetically modified cells to determine baseline levels of hemoglobin in the subject. In certain embodiments, hemoglobin, neutrophil and/or platelet levels in the subject after administration of the genetically modified cells increase or remain stable as compared to baseline levels for weeks or months after administration. Optionally, the subject may receive one or more PRBC transfusions prior to and/or after administration of the genetically modified cells. In any of the methods described herein, after administration of the composition to the subject, the need for additional therapies such as a bone marrow transplant, blood component, iron chelation, and/or therapy PRBC transfusions in the subject are reduced or eliminated, for example within about 1 to 30 or more days, including 1-20 days. The cells and subject may also be monitored before and/or after administration for example to determine the indel profile of cells isolated from peripheral blood samples, bone marrow aspirates, or other tissue sources in comparison with the indel profile of the infused cells to in order to monitor stability of the graft in the subject.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. The term “zinc finger nuclease” includes one ZFN as well as a pair of ZFNs (the members of the pair are referred to as “left and right” or “first and second” or “pair”) that dimerize to cleave the target gene.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. Nos. 8,586,526 and 9,458,205. The term “TALEN” includes one TALEN as well as a pair of TALENs (the members of the pair are referred to as “left and right” or “first and second” or “pair”) that dimerize to cleave the target gene. Zinc finger and TALE binding domains can be “engineered” to bind to a predetermined nucleotide sequence, for example via engineering (altering one or more amino acids) of the recognition helix region of a naturally occurring zinc finger or TALE protein. Therefore, engineered DNA binding proteins (zinc fingers or TALES) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 8,568,526; 6,140,081; 6,453,242; and 6,534,261; see also International Patent Publication Nos. WO 98/53058; WO 98/53059; WO 98/53060; WO 02/016536; and WO 03/016496.

A “selected” zinc finger protein or TALE is a protein not found in nature whose production results primarily from an empirical process such as phage display, interaction trap or hybrid selection. See e.g., U.S. Pat. Nos. 8,586,526; 5,789,538; 5,925,523; 6,007,988; 6,013,453; 6,200,759; and International Patent Publication Nos. WO 95/19431; WO 96/06166; WO 98/53057; WO 98/54311; WO 00/27878; WO 01/60970; WO 01/88197 and WO 02/099084.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break in the target sequence (e.g., cellular chromatin) at a predetermined site, and a “donor” polynucleotide, having homology to the nucleotide sequence in the region of the break, can be introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, additional pairs of zinc-finger or TALEN proteins can be used for additional double-stranded cleavage of additional target sites within the cell.

In certain embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In any of the methods described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence and is inserted into the genome by non-homologous recombination mechanisms.

Any of the methods described herein can be used for partial or complete inactivation of one or more target sequences in a cell by targeted integration of donor sequence that disrupts expression of the gene(s) of interest. Cell lines with partially or completely inactivated genes are also provided.

Furthermore, the methods of targeted integration as described herein can also be used to integrate one or more exogenous sequences. The exogenous nucleic acid sequence can comprise, for example, one or more genes or cDNA molecules, or any type of coding or non-coding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded. cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain another engineered cleavage half-domain). See, U.S. Pat. Nos. 7,888,121; 7,914,796; 8,034,598; and 8,823,618, incorporated herein by reference in their entireties.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “disease associated gene” is one that is defective in some manner in a monogenic disease. Non-limiting examples of monogenic diseases include severe combined immunodeficiency, cystic fibrosis, lysosomal storage diseases (e.g., Gaucher's, Hurler's Hunter's, Fabry's, Neimann-Pick, Tay-Sach's, etc.), sickle cell anemia, and thalassemia.

The “blood brain barrier” is a highly selective permeability barrier that separates the circulating blood from the brain in the central nervous system. The blood brain barrier is formed by brain endothelial cells which are connected by tight junctions in the CNS vessels that restrict the passage of blood solutes. The blood brain barrier has long been thought to prevent the uptake of large molecule therapeutics and prevent the uptake of most small molecule therapeutics (Pardridge (2005) NeuroRx 2(1): 3-14).

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of fusion molecules include, but are not limited to, fusion proteins (for example, a fusion between a protein DNA-binding domain and a cleavage domain), fusions between a polynucleotide DNA-binding domain (e.g., sgRNA) operatively associated with a cleavage domain, and fusion nucleic acids (for example, a nucleic acid encoding the fusion protein).

Expression of a fusion protein in a cell can result from delivery of the fusion protein to the cell or by delivery of a polynucleotide encoding the fusion protein a cell, wherein the polynucleotide is transcribed, and the transcript is translated, to generate the fusion protein. Trans-splicing, polypeptide cleavage and poly-peptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP or TALEN as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., stem cells, or precursor cells). The term “stem cells” or “precursor cells” refer to pluripotent and multipotent stem cells, including but not limited to hematopoietic stem cells, which are also referred to as hematopoietic progenitor stem cells (HPSC) or hematopoietic stem cell/precursor cells (HSC/PC).

“Red Blood Cells” (RBCs) or erythrocytes are terminally differentiated cells derived from hematopoietic stern cells. They lack a nuclease and most cellular organelles. RBCs contain hemoglobin to carry oxygen from the lungs to the peripheral tissues. In fact, 33% of an individual RBC is hemoglobin. They also carry CO2 produced by cells during metabolism out of the tissues and back to the lungs for release during exhale. RBCs are produced in the bone marrow in response to blood hypoxia which is mediated by release of erythropoietin (EPO) by the kidney. EPO causes an increase in the number of proerythroblasts and shortens the time required for full RBC maturation. After approximately 120 days, since the RBC do not contain a nucleus or any other regenerative capabilities, the cells are removed from circulation by either the phagocytic activities of macrophages in the liver, spleen and lymph nodes (˜90%) or by hemolysis in the plasma (˜10%). Following macrophage engulfment, chemical components of the RBC are broken down within vacuoles of the macrophages due to the action of lysosomal enzymes.

“Secretory tissues” are those tissues in an animal that secrete products out of the individual cell into a lumen of some type which are typically derived from epithelium. Examples of secretory tissues that are localized to the gastrointestinal tract include the cells that line the gut, the pancreas, and the gallbladder. Other secretory tissues include the liver, tissues associated with the eye and mucous membranes such as salivary glands, mammary glands, the prostate gland, the pituitary gland and other members of the endocrine system. Additionally, secretory tissues include individual cells of a tissue type which are capable of secretion.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP or TALE DNA-binding domain is fused to an activation domain, the ZFP or TALE DNA-binding domain and the activation domain are in operative linkage if, in the fusion polypeptide, the ZFP or TALE DNA-binding domain portion is able to bind its target site and/or its binding site, while the activation domain is able to up-regulate gene expression. When a fusion polypeptide in which a ZFP or TALE DNA-binding domain is fused to a cleavage domain, the ZFP or TALE DNA-binding domain and the cleavage domain are in operative linkage if, in the fusion polypeptide, the ZFP or TALE DNA-binding domain portion is able to bind its target site and/or its binding site, while the cleavage domain is able to cleave DNA in the vicinity of the target site.

A “functional” protein, polypeptide or nucleic acid includes any protein, polypeptide or nucleic acid that provides the same function as the wild-type protein, polypeptide or nucleic acid. A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods fur determining protein function are well-known, For example, the DNA-binding function of a polypeptide can be determined, for example, by filter-binding, electrophoretic mobility-shift, or immunoprecipitation assays. DNA cleavage can be assayed by gel electrophoresis. See, Ausubel et al., supra. The ability of a protein to interact with another protein can be determined, for example, by co-immunoprecipitation, two-hybrid assays or complementation, both genetic and biochemical. See, for example, Fields et at (1989) Nature 340:245-246; U.S. Pat. No. 5,585,245 and International Patent Publication No. WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

The terms “subject” and “patient” are used interchangeably and refer to mammals such as human subjects and non-human primates, as well as experimental animals such as rabbits, dogs, cats, rats, mice, and other animals. Accordingly, the term “subject” or “patient” as used herein means any mammalian subject or patient to which the altered cells of the invention and/or proteins produced by the altered cells of the invention can be administered. Subjects of the present invention include those having β-thalassemia disorder.

Generally, the subject or subject is eligible for treatment for β-thalassemia, For the purposes herein, such eligible subject or subject is one who is experiencing, has experienced, or is likely to experience, one or more signs, symptoms or other indicators of β-thalassemia; has been diagnosed with β-thalassemia, whether, for example, newly diagnosed, and/or is at risk for developing β-thalassemia. One suffering from or at risk for suffering from β-thalassemia may optionally be identified as one who has been screened for abnormally low levels of hemoglobin in their blood or plasma.

As used herein, “treatment” or “treating” is an approach for obtaining beneficial or desired results including clinical results. For purposes of this invention, beneficial or desired clinical results include, but are not limited to, one or more of the following: decreasing one or more symptoms resulting from the disease, diminishing the extent of the disease, stabilizing the disease (e.g., preventing or delaying the worsening of the disease), delay or slowing the progression of the disease, ameliorating the disease state, decreasing the dose of one or more other medications required to treat the disease, and/or increasing the quality of life.

As used herein, “delaying” or “slowing” the progression of β-thalassemia means to prevent, defer, hinder, slow, retard, stabilize, and/or postpone development of the disease. This delay can be of varying lengths of time, depending on the history of the disease and/or individual being treated.

As used herein, “at the time of starting treatment” refers to the time period at or prior to the first exposure to an β-thalassemia therapeutic composition such as the compositions of the invention. In some embodiments, “at the time of starting treatment” is about any of one year, nine months, six months, three months, second months, or one month prior to a β-thalassemia drug. In some embodiments, “at the time of starting treatment” is immediately prior to coincidental with the first exposure to an β-thalassemia therapeutic composition.

As used herein, “based upon” includes (1) assessing, determining, or measuring the subject characteristics as described herein (and preferably selecting a subject suitable for receiving treatment; and (2) administering the treatment(s) as described herein.

A “symptom” of β-thalassemia is any phenomenon or departure from the normal in structure, function, or sensation, experienced by the subject and indicative of β-thalassemia.

“Transfusion dependent β-thalassemia” (TDT) subjects require regular infusions (transfusions) of PRBC and other blood products to maintain hemoglobin levels >9 to 10 g/dL. TDT is a severe, progressive type ofβ-thalassemia characterized by severe anemia, lifelong transfusion dependence, unavoidable iron overload, serious comorbidities, and shorter lifespan compared with the general population. Patients with TDT require lifelong supportive care with regular blood transfusions—typically given every 2 to 5 weeks—to mitigate anemia and enable survival. Therapeutic levels, including levels that reduce or eliminate the need for blood transfusions may be above 2-10 or more g/dL (including 2, 5, 6, 7, 8, 9, 10 or more g/dL), optionally at least about 5 to 7 or more g/dL for transfusion independence.

Chronic transfusions lead to unavoidable iron overload that can result in significant damage to vital organs. Therefore, patients with TDT need continuous and rigorous monitoring of iron burden and must regularly take medications to remove excess iron, a process called iron chelation.

The term “supportive surgery” refers to surgical procedures that may be performed on a subject to alleviate symptoms that may be associated with a disease.

The term “immunosuppressive agent” as used herein for adjunct therapy refers to substances that act to suppress or mask the immune system of the mammal being treated herein. This would include substances that suppress cytokine production, down regulate or suppress self-antigen expression, or mask the MHC antigens. Examples of such agents include 2-amine-6-aryl-5-substituted pyrimidines (see, U.S. Pat. No. 4,665,077); nonsteroidal anti-inflammatory drugs (NSAIDUA); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); hydroxycloroquine; sulfasalazine; leflunomide; cytokine or cytokine receptor antagonists including anti-interferon-alpha, beta, or -gamma antibodies, anti-tumor necrosis factor-alpha antibodies (infliximab or adalimumab), anti-TNF-alpha immunoahesin (etanercept), anti-tumor necrosis factor-beta antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (International Patent Publication No. WO 90/08187 published Jul. 26, 1990); streptokinase; TGF-beta; streptodornase; RNA or DNA from the host; FK506; RS-61443; deoxyspergualin; rapamycin; T-cell receptor (Cohen et al., U.S. Pat. No. 5,114,721); T-cell receptor fragments (Offner et al. (1991) Science 251:430-432; International Patent Publication No. WO 90/11294; Janeway (1989) Nature 341:482; and International Patent Publication No. WO 91/01133); and T cell receptor antibodies such as T10B9.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone), dexamethasone, glucocorticoid and betamethasone.

“Iron chelation” is a type of therapy to remove excess iron from the body. Each unit of blood given in a transfusion comprises about 250 milligrams of iron, and the body cannot excrete it except in small (˜1 mg) amounts that are lost in skin and perspiration. Excess iron is trapped in the tissues of vital organs, such as the anterior pituitary, heart, liver, pancreas and joints. When the iron reaches toxic levels, damage can result in diseases such as diabetes, cirrhosis, osteoarthritis, heart attack, and hormone imbalances. Hypothyroidism, hypogonadism, infertility, impotence and sterility can result from these hormone imbalances. If not addressed, excess iron can result in complete organ failure and death. Iron reduction is accomplished with chelation therapy, which is the removal of iron pharmacologically with an iron-chelating agent such as desferrioxamine, (brand name Desferal or Jadenu®) or deferasirox, brand name Exjade®.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

A “label” is used herein to refer to information customarily included with commercial packages of pharmaceutical formulations including containers such as vials and package inserts, as well as other types of packaging.

It is to be understood that one, some, or all of the properties of the various embodiments described herein may be combined to form other embodiments of the present invention. These and other aspects of the invention will become apparent to one of skill in the art.

Nucleases

The methods described herein can make use of one or more nucleases for targeted knockout of the BCL11A erythroid enhancer. Non-limiting examples of nucleases include ZFNs, TALENs, homing endonucleases, CRISPR/Cas and/or Ttago guide RNAs, that are useful for in vivo cleavage of a donor molecule carrying a transgene and nucleases for cleavage of the genome of a cell such that the transgene is integrated into the genome in a targeted manner. See, e.g., U.S. Pat. Nos. 10,435,677; 10,072,066; 9,957,501; 9,963,715; 9,650,648; and U.S. Patent Publication Nos. 2019/0177709; 2018/0111975; and 2015/0132269. In certain embodiments, one or more of the nucleases are naturally occurring. In other embodiments, one or more of the nucleases are non-naturally occurring, i.e., engineered in the DNA-binding molecule (also referred to as a DNA-binding domain) and/or cleavage domain. For example, the DNA-binding domain of a naturally-occurring nuclease may be altered to bind to a selected target site (e.g., a ZFP, TALE and/or sgRNA of CRISPR/Cas that is engineered to bind to a selected target site). In other embodiments, the nuclease comprises heterologous DNA-binding and cleavage domains (e.g., zinc finger nucleases; TAL-effector domain DNA binding proteins; meganuclease DNA-binding domains with heterologous cleavage domains). In other embodiments, the nuclease comprises a system such as the CRISPR/Cas of Ttago system.

A. DNA-Binding Domains

In certain embodiments, the composition and methods described herein employ a meganuclease (homing endonuclease) DNA-binding domain for binding to the donor molecule and/or binding to the region of interest in the genome of the cell. Naturally-occurring meganucleases recognize 15-40 base-pair cleavage sites and are commonly grouped into four families: the LAGLIDADG family (SEQ ID NO: 17), the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22:1125-1127; Jasin (1996) Trends Genet. 12:224-228; Gimble et al. (1996) J. Mol Biol. 263:163-180; Argast et al. (1998) J. Mol. Biol. 280:345-353 and the New England Biolabs catalogue.

In certain embodiments, the methods and compositions described herein make use of a nuclease that comprises an engineered (non-naturally occurring) homing endonuclease (meganuclease). The recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort et al. (1997) Nucleic Acids Res. 25; 3379-3388; Dujon et al. (1989) Gene 82:115-118; Perler et al. (1994) Nucleic Acids Res. 22:1125 1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952.-2962; Ashworth et al. (2006) Nature 441:656-659; Paques et al. (2007) Current Gene Therapy 7:49-66; U.S. Patent Publication No. 2007/0117128. The DNA-binding domains of the homing endonucleases and meganucleases may be altered in the context of the nuclease as a whole (i.e., such that the nuclease includes the cognate cleavage domain) or may be fused to a heterologous cleavage domain.

In other embodiments, the DNA binding domain of one or more of the nucleases used in the methods and compositions described herein comprises a naturally occurring or engineered (non-naturally occurring) TAL effector DNA binding domain. See, e.g., U.S. Pat. No. 8,586,526, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like (TAL) effectors which mimic plant transcriptional activators and manipulate the plant transcriptome (see Kay et al. (2007) Science 318:648-651). These proteins contain a DNA binding domain and a transcriptional activation domain. One of the most well characterized TAL-effectors is AvrBs3 from Xanthomonas campestgris pv. Vesicatoria (see Bonas et al. (1989) Mol Gen Genet 218: 127-436 and International Patent Publication No. WO 2010/079430). TAL-effectors contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack et al. (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See, Heuer et al. (2007) Appl and Envir Micro 73(13):4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas. See, e.g., U.S. Pat. No. 8,586,526, incorporated by reference in its entirety herein.

Specificity of these TAL, effectors depends on the sequences found in the tandem repeats. The repeated sequence comprises approximately 102 bp and the repeats are typically 91-100% homologous with each other (Bonas et al., ibid). Polymorphism of the repeats is usually located at positions 12 and 13 and there appears to be a one-to-one correspondence between the identity of the hypervariable diresidues (RVDs) at positions 12 and 13 with the identity of the contiguous nucleotides in the TAL-effector's target sequence (see, Moscou and Bogdanove (2009) Science 326:1501 and Boch et al. (2009) Science 326:15094512). Experimentally, the natural code for DNA recognition of these TAL-effectors has been determined such that an HD sequence at positions 12 and 13 leads to a binding to cytosine (C), NG binds to T, NI to A, C, G or T, NN binds to A or U, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch et al., ibid). Engineered TAL proteins have been linked to a FokI cleavage half domain to yield a TAL effector domain nuclease fusion (TALEN) exhibiting activity in a yeast reporter assay (plasmid-based target). See, e.g., U.S. Pat. No. 8,586,526; Christian et al. (2010) Genetics epub 10.1534/genetics.110.120717).

In certain embodiments, the DNA binding domain of one or more of the nucleases used for in vivo cleavage and/or targeted cleavage of the genome of a cell comprises a zinc finger protein. Preferably, the zinc finger protein is non-naturally occurring in that it is engineered to bind to a target site of choice. See, for example, See, for example, Beerli et al. (2002) Nature Biotechnol, 20:135-141; Pabo et at (2001) Ann. Rev. Biochem. 70:313-340; Isalan et al. (2001) Nature Biotechnol, 19;656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; and 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197. In addition, enhancement of binding specificity for zinc finger binding domains has been described, for example, in co-owned International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 8,772,453; 6,479,626; 6,903,1.85; and 7,153,949 for exemplary linker sequences. The proteins described herein may include any combination of suitable linkers between the individual zinc fingers of the protein.

Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences that are 6 or more amino acids in length. The proteins described herein may include any combination of suitable linkers between the individual zinc fingers of the protein.

The zinc finger nuclease may comprise a ZFN pair (comprising left and right ZFNs) in which each ZFN pair comprises a nuclease (cleavage domain) and a ZFP targeted to BCL11A. See, e.g., U.S. Pat. Nos. 9,963,715; 9,650,648; U.S. Patent Publication Nos. 2015/0132269 and 2018/0111975. In certain embodiments, the ZFN pair of the mRNAs specifically modifies BCL11A (e.g., the +58 enhancer region) as compared to any other loci (off-target) and/or as compared to other BCL11A targeted nucleases (e.g., ZFNs without modifications to the backbone, which modifications are described in U.S. Pat. No. 10,563,184). Thus, cells produced using the mRNAs described herein are specifically modified at the BCL11A locus, including in which less than 10% (0 to 10% of any value therebetween), preferably less than 5% (0 to 5% or any value therebetween), even more preferably less than 1% of the cells (0 to 1% or any value therebetween) and even more preferably less than 0.5% (0 to 1% or any value therebetween) of the genetically modified cells include genetic modifications made by the mRNA(s) outside the BCL11A locus. See, e.g., U.S. Pat. No. 10,563,184. These cells may include additional modifications, for example inactivation of HLA genes.

In certain embodiments, the DNA-binding domain of the nuclease is part of a CRISPR/Cas nuclease system, including, for example a single guide RNA (sgRNA). See, e.g., U.S. Pat. No. 8,697,359 and U.S. Patent Publication No. 2015/0056705. The CRISPR (clustered regularly interspaced short palindromic repeats) locus, which encodes RNA components of the system, and the Cas (CRISPR-associated) locus, which encodes proteins (Jansen et al. (2002) Mol. Microbiol. 43:1565-1575; Makarova et al. (2002) Nucleic Acids Res. 30:482-496; Makarova et al. (2006) Biol. Direct 1:7; Haft et al. (2005) PLoS Comput. Biol. 1:e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In some embodiments, the CRISPR-Cpf1 system is used. The CRISPR-Cpf1 system, identified in Francisella spp., is a class 2 CRISPR-Cas system that mediates robust DNA interference in human cells. Although functionally conserved, Cpf1 and Cas9 differ in many aspects including in their guide RNAs and substrate specificity (see, Fagerlund et al. (2015) Genom Bio 16:251). A major difference between Cas9 and Cpf1 proteins is that Cpf1 does not utilize tracrRNA, and thus requires only a crRNA. The FnCpf1 crRNAs are 42-44 nucleotides long (19-nucleotide repeat and 23-25-nucleotide spacer) and contain a single stem-loop, which tolerates sequence changes that retain secondary structure. In addition, the Cpf1 crRNAs are significantly shorter than the ˜100-nucleotide engineered sgRNAs required by Cas9, and the PAM requirements for FnCpf1 are 5′-TTN-3′ and 5′-CTA-3′ on the displaced strand. Although both Cas9 and Cpf1 make double strand breaks in the target DNA, Cas9 uses its RuvC- and HNH-like domains to make blunt-ended cuts within the seed sequence of the guide RNA, whereas Cpf1 uses a RuvC-like domain to produce staggered cuts outside of the seed. Because Cpf1 makes staggered cuts away from the critical seed region, NHEJ will not disrupt the target site, therefore ensuring that Cpf1 can continue to cut the same site until the desired HDR recombination event has taken place. Thus, in the methods and compositions described herein, it is understood that the term “Cas” includes both Cas9 and Cfp1 proteins. Thus, as used herein, a “CRISPR/Cas system” refers both CRISPR/Cas and/or CRISPR/Cfp1 systems, including both nuclease, nickase and/or transcription factor systems.

In some embodiments, other Cas proteins may be used. Some exemplary Cas proteins include Cas9, Cpf1 (also known as Cas12a), C2c1, C2c2 (also known as Cas13a), C2c3, Cas1, Cas2, Cas4, CasX and CasY; and include engineered and natural variants thereof (Burstein et al. (2017) Nature 542:237-241) for example HF1/spCas9 (Kleinstiver et al. (2016) Nature 529: 490-495; Cebrian-Serrano and Davies (2017) Mamm Genome (2017) 28(7):247-261); split Cas9 systems (Zetsche et al. (2015) Nat Biotechnol 33(2):139-142), trans-spliced Cas9 based on an intein-extein system (Troung et al. (2015) Nucl Acid Res 43(13):6450-8); mini-SaCas9 (Ma et al. (2018) ACS Synth Biol 7(4):978-985). Thus, in the methods and compositions described herein, it is understood that the term “Cas” includes all Cas variant proteins, both natural and engineered.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein. Additional non-limiting examples of RNA guided nucleases that may be used in addition to and/or instead of Cas proteins include Class 2 CRISPR proteins such as Cpf1. See, e.g., Zetsche et al. (2015) Cell 163:1-13.

In some embodiments, the DNA binding domain is part of a TtAgo system (see, Swarts et al. (2014) Nature 507(7491):258-261; Swarts et al. (2012) PLoS One 7(4):e35888 and Sheng et al. (2014) Proc. Natl. Acad. Sci. U.S.A. 111(2):652-657). In eukaryotes, gene silencing is mediated by the Argonaute (Ago) family of proteins. In this paradigm, Ago is bound to small (19-31 nucleotide) RNAs. This protein-RNA silencing complex recognizes target RNAs via Watson-Crick base pairing between the small RNA and the target and endonucleolytically cleaves the target RNA (Vogel (2014) Science 344:972-973). In contrast, prokaryotic Ago proteins bind to small single-stranded DNA fragments and likely function to detect and remove foreign (often viral) DNA (Yuan et al. (2005) Mol. Cell 19:405; Olovnikov et al. (2013) Mol. Cell 51:594; Swarts et al., ibid). Exemplary prokaryotic Ago proteins include those from Aquifex aeolicus, Rhodobacter sphaeroides, and Thermus thermophilus.

One of the most well-characterized prokaryotic Ago protein is the one from T. thermophilus (TtAgo; Swarts et al., ibid). TtAgo associates with either 15 nucleotides or 13-25 nucleotide single-stranded DNA fragments with 5′ phosphate groups. This “guide DNA” bound by TtAgo serves to direct the protein-DNA complex to bind a Watson-Crick complementary DNA sequence in a third-party molecule of DNA. Once the sequence information in these guide DNAs has allowed identification of the target DNA, the TtAgo-guide DNA complex cleaves the target DNA. Such a mechanism is also supported by the structure of the TtAgo-guide DNA complex while bound to its target DNA (Sheng et al., ibid.), Ago from Rhodobacter sphaeroides (RsAgo) has similar properties (Olovnikov et al., ibid).

Exogenous guide DNAs of arbitrary DNA sequence can be loaded onto the TtAgo protein (Swarts et al., ibid.). Since the specificity of TtAgo cleavage is directed by the guide DNA, a TtAgo-DNA complex formed with an exogenous, investigator-specified guide DNA will therefore direct TtAgo target DNA cleavage to a complementary investigator-specified target DNA. In this way, one may create a targeted double-strand break in DNA. Use of the TtAgo-guide DNA system (or orthologous Ago-guide DNA systems from other organisms) allows for targeted cleavage of genomic DNA within cells. Such cleavage can be either single- or double-stranded. For cleavage of mammalian genomic DNA, it would be preferable to use of a version of TtAgo codon optimized for expression in mammalian cells. Further, it might be preferable to treat cells with a TtAgo-DNA complex formed in vitro where the TtAgo protein is fused to a cell-penetrating peptide. Further, it might be preferable to use a version of the TtAgo protein that has been altered via mutagenesis to have improved activity at 37 degrees Celsius. TtAgo-RNA-mediated DNA cleavage could be used to affect a panoply of outcomes including gene knock-out, targeted gene addition, gene correction, targeted gene deletion using techniques standard in the art for exploitation of DNA breaks.

Thus, the nuclease comprises a DNA-binding domain in that specifically binds to a target site in any gene into which it is desired to insert a donor (transgene).

In certain embodiments the DNA-binding domains bind to albumin, e.g., DNA-binding domains of the ZFPs designated SBS-47171 and SBS-47898. See, e.g., U.S. Patent Publication No. 2015/0159172.

B. Cleavage Domains

Any suitable cleavage domain can be associated with (e.g., operatively linked) to a DNA-binding domain to form a nuclease. For example, ZFP DNA binding domains have been fused to nuclease domains to create ZFNs—a functional entity that is able to recognize its intended nucleic acid target through its engineered (ZFP) DNA binding domain and cause the DNA to be cut near the ZFP binding site via the nuclease activity. See, e.g., Kim et al. (1996) Proc Natl Acad Sci USA 93(3):1156-1160. More recently, ZFNs have been used for genome modification in a variety of organisms. See, for example, U.S. Patent Publication Nos. 2003/0232410; 2005/0208489; 2005/0026157; 2005/0064474; 2006/0188987; 2006/0063231; and International Patent Publication No. WO 07/014275. Likewise, TALE DNA-binding domains have been fused to nuclease domains to create TALENs. See, e.g., U.S. Pat. No. 8,586,526. CRISPR/Cas nuclease systems comprising single guide RNAs (sgRNAs) that bind to DNA and associate with cleavage domains (e.g., Cas domains) to induce targeted cleavage have also been described. See, e.g., U.S. Pat. Nos. 8,697,359 and 8,932,814 and U.S. Patent Publication No. 2015/0056705.

As noted above, the cleavage domain may be heterologous to the DNA-binding domain, for example a zinc finger DNA-binding domain and a cleavage domain from a nuclease or a TALEN DNA-binding domain and a cleavage domain from a nuclease; a sgRNA DNA-binding domain and a cleavage domain from a nuclease (CRISPR/Cas); and/or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides, However, any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is FokI. This particular enzyme is active as a dimer. Bitinaite et al. (1998) Proc. Natl. Acad. Sci. USA 95:10,570-10,575. Accordingly, for the purposes of the present disclosure, the portion of the FokI enzyme used in the disclosed fusion proteins is considered a cleavage half-domain. Thus, for targeted double-stranded cleavage and/or targeted replacement of cellular sequences using zinc finger-FokI fusions, two fusion proteins, each comprising a FokI cleavage half-domain, can be used to reconstitute a catalytically active cleavage domain. Alternatively, a single polypeptide molecule containing a zinc finger binding domain and two FokI cleavage half-domains can also be used. Parameters for targeted cleavage and targeted sequence alteration using zinc finger-FokI fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in U.S. Pat. No. 7,888,121, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Pat. Nos. 8,772,453; 8,623,618; 8,409,861; 8,034,598; 7,914,796; and 7,888,121, the disclosures of all of which are incorporated by reference in their entireties herein. Amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains.

Exemplary engineered cleavage half-domains of FokI that form obligate heterodimers include a pair in which a first cleavage half-domain includes mutations at amino acid residues at positions 490 and 538 of FokI and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K).

Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished, U.S. Pat. Nos. 7,914,796 and 8,034,598, the disclosures of which are incorporated by reference in their entireties. In certain embodiments, the engineered cleavage half-domain comprises mutations at positions 486, 499 and 496 (numbered relative to wild-type FokI), for instance mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). See, e.g., U.S. Pat. No. 8,772,453. In other embodiments, the engineered cleavage half domain comprises the “Sharkey” and/or “Sharkey mutations” (see, Guo et al. (2010) J. Mol. Biol. 400(1):96-107).

Engineered cleavage half-domains described herein can be prepared using any suitable method, for example, by site-directed mutagenesis of wild-type cleavage half-domains (FokI) as described in U.S. Pat. Nos. 7,888,121; 7,914,796; 8,034,598; and 8,623,618.

Alternatively, nucleases may be assembled in vivo at the nucleic acid target site using so-called “split-enzyme” technology (see, e.g., U.S. Patent Publication No. 2009/0068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in U.S. Pat. No. 8,563,314. Expression of the nuclease may be under the control of a constitutive promoter or an inducible promoter, for example the galactokinase promoter which is activated (de-repressed) in the presence of raffinose and/or galactose and repressed in presence of glucose.

The Cas9 related CRISPR/Cas system comprises two RNA non-coding components; tracrRNA and a pre-crRNA array containing nuclease guide sequences (spacers) interspaced by identical direct repeats (DRs). To use a CRISPR/Cas system to accomplish genome engineering, both functions of these RNAs must be present (see, Cong et al. (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA). (see, Jinek et al. (2012) Science 337:816-821, Jinek et al. (2013) eLife 2:e00471 and Cong, ibid).

The nuclease(s) as described herein may make one or more double-stranded and/or single-stranded cuts in the target site. In certain embodiments, the nuclease comprises a catalytically inactive cleavage domain (e.g., FokI and/or Cas protein). See, e.g., U.S. Pat. Nos. 9,200,266; 8,703,489 and Guillinger et al. (2014) Nature Biotech. 32(6):577-582. The catalytically inactive cleavage domain may, in combination with a catalytically active domain act as a nickase to make a single-stranded cut. Therefore, two nickases can be used in combination to make a double-stranded cut in a specific region. Additional nickases are also known in the art, for example, McCaffery et al. (2016) Nucleic Acids Res. 44(2):e11. doi: 10.1093/nar/gkv878. Epub 2015 Oct. 19.

Thus, any nuclease comprising a DNA-binding domain and cleavage domain can be used. In certain embodiments, the nuclease comprises a ZFN made up of first and second (also referred to as left and right ZFNs), for example a ZFN comprising a first ZFN comprising a ZFP designated SBS-63014 and a cleavage domain and a second ZFN comprising a ZFP designated SBS-65722 and a cleavage domain. In certain embodiments, the left and right (first and second) ZFNs of the ZFN are carried on the same vector and in other embodiments, the paired components of the ZFN are carried on different vectors, for example two mRNAs vectors as shown in Example 1, one designated SB-mRENH1 mRNA (an mRNA encoding the ZFN comprising the ZFP designated 63014) and the other designated SB-mRENH2 mRNA (an mRNA encoding the ZFN comprising the ZFP designated 65722).

Target Sites

As described in detail above, DNA domains can be engineered to bind to any sequence of choice in a locus, for example an albumin or other safe-harbor gene. An engineered DNA-binding domain can have a novel binding specificity, compared to a naturally-occurring DNA-binding domain. Engineering methods include, but are not limited to, rational design and various types of selection. Rational design includes, for example, using databases comprising triplet (or quadruplet) nucleotide sequences and individual (e.g., zinc finger) amino acid sequences, in which each triplet or quadruplet nucleotide sequence is associated with one or more amino acid sequences of DNA binding domain which bind the particular triplet or quadruplet sequence. See, for example, co-owned U.S. Pat. Nos. 6,453,242 and 6,534,261, incorporated by reference herein in their entireties. Rational design of TAL-effector domains can also be performed. See, e.g., U.S. Patent Publication No. 2011/0301073.

Exemplary selection methods applicable to DNA-binding domains, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237.

Selection of target sites; nucleases and methods for design and construction of fusion proteins (and polynucleotides encoding same) are known to those of skill in the art and described in detail in U.S. Patent Publication Nos. 2005/0064474 and 2006/0188987, incorporated by reference in their entireties herein.

In addition, as disclosed in these and other references, DNA-binding domains (e.g., multi-fingered zinc finger proteins) may be linked together using any suitable linker sequences, including for example, linkers of 5 or more amino acids. See, e.g., U.S. Pat. Nos. 6,479,626; 6,903,185; and 7,153,949 for exemplary linker sequences of 6 or more amino acids in length. The proteins described herein may include any combination of suitable linkers between the individual DNA-binding domains of the protein, See, also, U.S. Pat. No. 8,586,526.

In certain embodiments, the target site(s) for the DNA-binding domain(s) (is)are within a BCL11A gene. See, e.g., U.S. Pat. Nos. 10,563,184; 9,963,715; 9,650,648; U.S. Patent Publication Nos. 2015/0132269; 2018/0111975; and 2019/0177709.

Compositions/Systems of the Invention

Described herein are modified autologous HSC/PC that are delivered to the subject to practice the methods according to certain embodiments. Two mRNAs encoding the right and left ZFN partners are delivered to the harvested HSC/PC which are targeted to the BCL11a erythroid enhancer sequence. In certain embodiments, the mRNAs include SB-mRENH1 and SB-mRENH2. In any of the methods described herein, the CD34+ HSC/PCs are harvested (e.g., apheresis) after mobilization in the subject by treating the subject with one or more doses of G-CSF and/or one or more doses of plerixafor prior to isolation and the mobilized cells. In certain embodiments, at least about 25×10⁶ CD34+ HSPCs/kg are harvested in total or per apheresis cycle and may be cultured for any length of time. The resulting genetically modified cells may be cultured and descendants thereof will include the specific BCL11A genetic modification (e.g., less than 1% of cells having off-target (non-BCL11A) modifications), but not necessarily the mRNA(s).

Cells comprising the BCL11A knockout are then infused into the subjects. Additional modifications, for example inactivation of HLA genes may be made in the specific BCL11A genetically modified cells.

Cells

Also provided herein are genetically modified cells, for example, HSC/PC comprising a targeted knockout of the BCL11A erythroid enhancer. The knockout is created by treating harvested HSC/PC with mRNAs encoding the right and left ZFN partners which when translated, will result in an active ZFN. The ZFN cleaves the BCL11A erythroid enhancer such that a double strand break in the DNA occurs. The cellular machinery repairs the double strand break using error-prone non-homologous end joining (NHEJ) which results in the insertion and deletion of nucleotides (indels) around the cleavage site.

Both autologous (e.g., subject-derived) and allogenic (healthy donor derived) HSC/PC can be used in the performance of the method.

The cells as described herein are useful in cell therapy for treating and/or preventing β-thalassemia disease in a subject with the disorder. In the case of modified stem cells, after infusion into the subject, in vivo differentiation of these precursors into cells expressing the functional protein (from the inserted donor) also occurs.

Pharmaceutical compositions comprising the cells as described herein are also provided. In addition, the cells may be cryopreserved prior to administration to a subject.

The cell populations (and compositions) described herein comprise genetically modified cells specifically at the BCL11A locus, including genetically modified cell populations in which less than 10% (0 to 10% of any value therebetween), preferably less than 5% (0 to 5% or any value therebetween), even more preferably less than 1% of the cells (0 to 1% or any value therebetween) and even more preferably less than 0.5% (0 to 1% or any value therebetween) of the cells include genetic modifications outside the BCL11A locus (but may include additional modifications such as inactivation of HLA markers).

Delivery

The ex vivo delivery of nucleases, polynucleotides encoding these nucleases, donor polynucleotides and compositions comprising the proteins and/or polynucleotides described herein may be delivered to the harvested HSC/PC by any suitable means.

Methods of delivering nucleases as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Nucleases and/or donor constructs as described herein may also be delivered using vectors containing sequences encoding one or more of the zinc finger, TAL-effector domain and/or Cas protein(s). Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties.

Conventional viral and non-viral based gene transfer methods can be used to introduce nucleic acids encoding nucleases and donor constructs in cells (e.g., mammalian cells) and target tissues. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see, Anderson (1992) Science 256:808-813; Nabel & Felgner (1993) TIBTECH 11:211-217; Mitani & Caskey (1993) TIBTECH 11:162-466; Dillon (1993) TIBTECH 11:167-175; Miller (1992) Nature 357:455-460; Van Brunt (1988) Biotechnology 6(10):1149-1154; Vigne (1995) Restorative Neurology and Neuroscience 8:35-36; Kremer & Perricaudet (1995) British Medical Bulletin 51(1):31-44; Haddada et al., in Current Topics in Microbiology and Immunology Doerfler and Böhm (eds.) (1995); and Yu et al. (1994) Gene Therapy 1:13-26.

Methods of non-viral delivery of nucleic acids include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Arnaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in U.S. Pat. Nos. 5,049,386; 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424, WO 91/16024.

The preparation of lipid:nucleic acid complexes, including targeted liposomes such as immunolipid complexes, is well known to one of skill in the art (see, e.g., Crystal (1995) Science 270:404-410; Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see, MacDiarmid et al. (2009) Nature Biotechnology 27(7):643).

The use of RNA or DNA viral based systems for the delivery of nucleic acids encoding engineered ZFPs take advantage of highly evolved processes for targeting a virus to specific cells in the body and trafficking the viral payload to the nucleus. Viral vectors can be used to treat cells in vitro and the modified cells are administered to subjects (ex vivo). Conventional viral based systems for the delivery of ZFPs include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long tern expression of the inserted transgene. Additionally, high transduction efficiencies have been measured in many different cell types and target tissues.

Recombinant adeno-associated virus vectors (rAAV) are a promising alternative gene delivery system based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al. (1998) Lancet 351(9117):1702-3; Kearns et al. (1996) Gene Ther. 9:748-55). Other AAV serotypes, including by non-limiting example, AAV1, AAV 3, AAV4, AAV5, AAV6, AAV8, AAV 8.2, AAV9 and AAV rh10 and pseudotyped AAV such as AAV2/8, AAV2/5 and AAV2/6 can also be used in accordance with the present invention. In some embodiments, AAV serotypes that are capable of crossing the blood brain barrier are used.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity. An example of the use of an Ad vector in a clinical trial involved polynucleotide therapy for anti-tumor immunization with intramuscular injection (Sterman et al. (1998) Hum. Gene Ther. 7:1083-9). Additional examples of the use of adenovirus vectors for gene transfer in clinical trials include Rosenecker et al. (1996) Infection 24(1):5-10; Sterman et al. (1998) Hum. Gene Ther. 9(7):1083-1089; Welsh et al. (1995) Hum. Gene Ther. 2:205-18; Alvarez et al. (1997) Hum. Gene Ther. 5:597-613; Topf et al. (1998) Gene Ther. 5:507-513; Sterman et al. (1998) Hum. Gene Ther. 7:1083-1089.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line, For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

Compositions comprising genetically modified cells as described herein may be delivered to a subject in any suitable manner, including by infusion. Prior to administration of composition comprising the genetically modified cells, the subject may be treated with (administered) one or more myeloablative condition agents one or more times, for example, busulfan administered: intravenously (IV) at between about 0.5 to 5 mg/kg for one or more times; IV at about 3.2 mg/kg/day; IV via central venous catheter for 4 days total dose of about 12.8 mg/kg prior to infusion on Days −6 through −3 before infusion of the composition comprising the genetically modified cells on Day 0; or IV once daily or every 6 hours.

Any dose of genetically modified cells can be used, for example, between about 3×106 cells/kg and about 20×106 cells/kg (e.g., where the cells are formulated with approximately 1.0-2.0×108 cells per bag at a concentration of approximately 1×107 cells/mL).

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

Formulations for both ex vivo and in vivo administrations include suspensions in liquid or emulsified liquids. The active ingredients often are mixed with excipients which are pharmaceutically acceptable and compatible with the active ingredient. Suitable excipients include, for example, water, saline, dextrose, glycerol, ethanol or the like, and combinations thereof. In addition, the composition may contain minor amounts of auxiliary substances, such as, wetting or emulsifying agents, pH buffering agents, stabilizing agents or other reagents that enhance the effectiveness of the pharmaceutical composition.

Applications

The methods of this invention contemplate the treatment and/or prevention of β-thalassemia. Treatment can comprise knock out of the BCL11A enhancer sequence in a cell to block the expression of the BCL11A protein. BCL11a protein is known to repress expression of fetal globin, so knock out of BCL11A will result in a lack of repression of the HbF gene. The methods and compositions of the invention also can be used in any circumstance wherein it is desired to knock out the BCL11A erythroid enhancer in a hematopoietic stem cell such that mature cells (e.g., RBCs) derived from these cells contain the therapeutic knockout. These stem cells can be differentiated in vitro or in vivo and may be derived from a universal donor type of cell which can be used for all subjects. Additionally, the cells may contain a transmembrane protein to traffic the cells in the body. Treatment can also comprise use of subject cells containing the therapeutic transgene where the cells are developed ex vivo and then introduced back into the subject. For example, HSC/PC containing a BCL11A erythroid enhancer knockout may be inserted into a subject via an autologous bone marrow transplant.

Thus, this technology may be of use in a condition where a subject has a mutation in their B-globin gene or a deficiency in its expression. Genetic defects in the sequences encoding the hemoglobin chains can be responsible for a group of diseases known as hemoglobinopathies that include sickle cell anemia and the beta thalassemias. In thalassemia minor, only one of the β globin alleles bears a mutation. Individuals will suffer from microcytic anemia, and detection usually involves lower than normal mean corpuscular volume (<80 fL). The alleles of subjects with thalassemia minor are β+/β or β0/β (where ‘β+’ refers to alleles that allow some amount of β chain formation to occur, ‘β’ refers to wild type β globin alleles, and ‘β0’ refers to β globin mutations associated with a complete absence of beta globin expression). Thalassemia intermedia subjects can often manage a normal life hut may need occasional transfusions, especially at times of illness or pregnancy, depending on the severity of their anemia. These patient's alleles can be β+/β+ or β0/β+. Thalassemia major occurs when both alleles have thalassemia mutations (β0/β0). This is severely microcytic and hypochromic anemia. Untreated, it causes anemia, splenomegaly and severe bone deformities and progresses to death before age 20. Treatment consists of periodic blood transfusion; splenectomy for splenomegaly and chelation of transfusion-caused iron overload. Bone marrow transplants are also being used for treatment of people with severe thalassemias if an appropriate donor can be identified, but this procedure can have significant risks. In the majority of patients with hemoglobinopathies, the genes encoding gamma globin remain present, but expression is relatively low due to normal gene repression occurring around parturition.

In some applications, provided herein is a method of improving or maintaining (slowing the decline) of thalassemia-related disease biomarkers in a human subject having β-thalassemia (e.g., β-thalassemia major (TDT) or β-thalassemia minor) as compared with a subject that has not been treated with the methods and compositions of the invention. In other applications, provided herein is a method of decreasing the need (dose level or frequency) for PRBC or other blood product infusions in a subject with β thalassemia as compared with the subject prior to treatment with the methods and compositions of the invention. In yet another aspect, provided herein is a method of reducing iron overload in a patient with β-thalassemia that occurs from chronic blood product infusions.

Thus, provided herein are methods of treating a beta-thalassemia (e.g., TDT) in a subject in need thereof by administering (e.g., by infusion) a genetically modified cell in which BCL11A is inactivated in the cell to the subject such that HbF production in the subject is increased and one or more clinical symptoms of β-thalassemia are decreased. The subjects with TDT that are treated may exhibit one or more of the following: (1) a change from baseline of clinical laboratory hemoglobin fractions (adult hemoglobin, HbA and fetal hemoglobin, HbF) in grams/dL plasma and/or percent HbF of total Hb; (2) alteration (e.g., to or near normal levels) of thalassemia-related disease biomarkers such biomarkers of iron metabolism; and/or levels of erythropoietin, haptoglobin and/or hepcidin; (3) reduction or elimination of symptoms in the subject associated with iron overload associated with baseline transfusion therapy, optionally wherein a decrease in endocrine dysfunction is assayed by measuring level and/or activity of thyroid hormones, IGF-1, morning cortisol, adrenocorticotropic hormone (ACTH), HbA1C, vitamin D, HbA, HbF, erythropoietin, haptoglobin, hepcidin, thyroid hormones, IGF-1, cortisol, ACTH and/or vitamin D in the subject; (4) reduction or elimination of the need for blood product infusions, including PRBC transfusions, platelet infusions, IVIG, plasma transfusion and/or granulocyte transfusion; (5) reduction or elimination of liver disease; (6) reduction or elimination of cardiac abnormalities; (7) reduction and/or elimination of osteoporosis and/or bone fractures and/or a change from baseline in bone mineral density; (8) reduction or elimination of atypical morphologies (e, g., hyperplasia) and/or the number of immature erythroid cells; and/or (9) a change from baseline (pre-treatment levels) in the number and percent of F cells.

The Karnofsky Performance Scale is a simple, widely-accepted tool for evaluating functional impairment in patients. Each subject will be evaluated and scored at the specified visit using the Karnofsky Performance Status Scale Definitions Rating Criteria. Subjects with a score on the Karnofsky Performance Scale ≤60 at the screening visit are not eligible to participate in this study. Change from baseline will be evaluated.

The genetically modified cells may be stem cells (e.g., CD34+ HSC/PC, ST-400) and may be autologous or allogeneic (e.g., isolated from healthy donors) and the allogeneic cells may be further modified (e.g., in addition to BCL11A inactivation), for example to remove one or more self-antigens (e.g., HLA complexes) to from the allogeneic cells, See, e.g., U.S. Pat. Nos. 8,945,868; 10,072,062; U.S. Patent Publication No. 2018/0362926. Autologous cells may be mobilized in the subject prior to modification ex vivo by treating the subject with one or more doses of G-CSF and/or one or more doses of plerixafor and the mobilized cells are harvested by one or more apheresis cycles. Optionally, at least about 25×10⁶ CD34+ HSPCs/kg are mobilized in the subject. The cells may be genetically modified to inactivate BCL11A using one or more nucleases, for example wherein the nucleases are introduced into the cell as mRNAs as disclosed herein (SEQ ID NO:15 and SEO ID NO:16). Following ex vivo genetic modification, the cells may be evaluated for insertions and/or deletions within BCL11A.

The subject to be treated may also be pre-treated with one or more myeloablative agents prior to administration of the genetically modified cells (e.g., 10 to 1 day before treatment), for example, via intravenous (IV) administration of busulfan is at between about 0.5 to 5 mg/kg (or any value therebetween) for one or more times; IV administration of busulfan is about 3.2 mg/kg/day; IV via central venous catheter for 4 days total dose of about 12.8 mg/kg prior to infusion on Days −6 through −3 before infusion of the modified HSPC on Day 0; or IV administration of busulfan is once daily (e.g., 4 doses) or every 6 hours (total of 16 doses). Any dose of genetically modified cells may be used, including but not limited to between about 3×106 cells/kg and about 20×106 cells/kg optionally wherein the cells are formulated in infusible cryomedia containing 10% DMSO. The cells may be formulated in any suitable container or packaging, for example in an infusion bag (e.g., comprising approximately 1.0-2.0×108 cells per bag at a concentration of approximately 1×107 cells/mL).

As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

The following Examples relate to exemplary embodiments of the present disclosure in which the nuclease comprises a zinc finger nuclease (ZFN) or TALEN. It will be appreciated that this is for purposes of exemplification only and that other nucleases or nuclease systems can be used, for instance homing endonucleases (meganucleases) with engineered DNA-binding domains and/or fusions of naturally occurring of engineered homing endonucleases (meganucleases) DNA-binding domains and heterologous cleavage domains and/or a CRISPR/Cas system comprising an engineered single guide RNA.

### EXAMPLES

### Example 1

### ZFN Design

The ZFN pair is made up of a 6-finger ZFN (encoded by mRNA SB-mRENH1) and a 5-finger ZFN (encoded by mRNA SB-mRENH2) that binds to a 33 base pair (combined) target site in the erythroid-specific enhancer of the human BCL11A gene at location chr2:60,495,250-60,495,290 in the GRCh38/hg38 assembly of the human genome. The preparation of the ZFN and polynucleotides encoding them is as follows: The SH-mRENH1 and SB-mRENH2 mRNAs are produced in vitro by methods known in the art. The mRNAs comprise sequences encoding the ZFN partners, and also comprise features such as nuclear localization sequences and peptides. Table 1 shows the helices associated with each partner ZFN (see U.S. Pat. No. 10,563,184; U.S. Patent Publication No. 2018/0087072):

TABLE 1
ZFN design

| SBS # | Design | | | | | | |
|---|---|---|---|---|---|---|---|
| (target | [Helix Sequence, SEQ ID] | | | | | | Linker |
| site, 5′- | [Mutations to finger backbone] | | | | | | Fok |
| 3′) | F1 | F2 | F3 | F4 | F5 | F6 | mutants |
| 63014 | DQSNLRA | RNFSLTMI | STGNLTN | TSGSLTR | DQSNLRA | AQCCLFH | L7c5 |
| aaAGCAACt | (SEQ ID | (SEQ ID | (SEQ ID | (SEQ ID | (SEQ ID | (SEQ ID | |
| GTTAGCTTG | NO: 5) | NO: 6) | NO: 7) | NO: 8) | NO: 5) | NO: 9) | |
| CACtagact | Qm5 | none | Qm5 | none | Qm5 | none | ELD |
| a (SEQ ID | | | | | | | |
| NO: 3) | | | | | | | |
| 65722 | RNDHRTT | QKABLIR | QKGTLGE | RGRDLSR | RRDNLHS | N/A | L0 |
| caCAGGCTC | (SEQ ID | (SEQ ID | (SEQ ID | (SEQ ID | (SEQ ID | | |
| CAGGAAGGg | NO: 10) | NO: 11) | NO: 12) | NO: 13) | NO: 14) | | |
| tttgqcctc | Qm5 | Qm5 | none | Qm5 | none | N/A | KKR |
| t (SEQ ID | | | | | | | K525S |
| NO: 4) | | | | | | | |

The complete nucleotide sequence for the SB-mRENH1 mRNA (1725 nucleotides) is shown below:

(SEQ ID NO: 15)
5′gggagacaagcuuugaauuacaagcuugcuuguucuuuuugcagaagcu
cagaauaaacgcucaacuuuggcagaucgaauucgccauggacuacaaaga
ccaugacggugauuauaaagaucaugacaucgauuacaaggaugacgauga
caagauggcccccaagaagaagaggaaggucggcauccacgggguacccgc
cgcuauggcugagaggcccuuccagugucgaaucugcaugcagaacuucag
ugaccaguccaaccugcgcgcccacauccgcacccacaccggcgagaagcc
uuuugccugugacauuugugggaggaaauuugcccgcaacuucucccugac
caugcauaccaagauacacacgggcagccaaaagcccuuccagugucgaau
cugcaugcagaacuucaguuccaccggcaaccugaccaaccacauccgcac
ccacaccggcgagaagccuuuugccugugacauuugugggaggaaauuugc
caccuccggcucccugacccgccauaccaagauacacacgcacccgcgcgc
cccgaucccgaagcccuuccagugucgaaucugcaugcagaacuucaguga
ccaguccaaccugcgcgcccacauccgcacccacaccggcgagaagccuuu
ugccugugacauuugugggaggaaauuugccgcccaguguugucuguucca
ccauaccaagauacaccugcggggauccaucagcagagccagaccacugaa
cccgcacccggagcuggaggagaagaaguccgagcugcggcacaagcugaa
guacgugccccacgaguacaucgagcugaucgagaucgccaggaacagcac
ccaggaccgcauccuggagaugaaggugauggaguucuucaugaaggugua
cggcuacaggggaaagcaccugggcggaagcagaaagccugacggcgccau
cuauacagugggcagccccaucgauuacggcgugaucguggacacaaaggc
cuacagcggcggcuacaaucugccuaucggccaggccgacgagauggagag
auacguggaggagaaccagacccgggauaagcaccucaaccccaacgagug
guggaagguguacccuagcagcgugaccgaguucaaguuccuguucgugag
cggccacuucaagggcaacuacaaggcccagcugaccaggcugaaccacau
caccaacugcaauggcgccgugcugagcguggaggagcugcugaucggcgg
cgagaugaucaaagccggcacccugacacuggaggaggugcggcgcaaguu
caacaacggcgagaucaacuucagaucuugauaacucgagucuagaagcuc
gcuuucuugcuguccaauuucuauuaaagguuccuuuguucccuaagucca
acuacuaaacugggggauauuaugaagggccuugagcaucuggauucugcc
uaauaaaaaacauuuauuuucauugcugcgcuagaagcucgcuuucuugcu
guccaauuucuauuaaagguuccuuuguucccuaaguccaacuacuaaacu
gggggauauuaugaagggccuugagcaucuggauucugccuaauaaaaaac
auuuauuuucauugcugcgggacauucuuaauuaaaaaaaaaaaaaaaaaa
aaaaaaaaaaaaaaaaaaaaaaaaaaaaaaaaaaaaaaaaaacuag

Further, the complete nucleotide sequence for SB-mRENH2 mRNA is shown below (1680 nucleotides):

(SEQ ID NO: 16)
5′gggagacaagcuugaauacaagcuugcuuguucuuuuugcagaagcuca
gaauaaacgcucaacuuuggcagaucgaauucgccuagagaucuggcggcg
gagagggcagaggaagucuucuaaccugcggugacguggaggagaaucccg
gcccuaggaccauggacuacaaagaccaugacggugauuauaaagaucaug
acaucgauuacaaggaugacgaugacaagauggcccccaagaagaagagga
aggucggcauucaugggguacccgccgcuauggcugagaggcccuuccagu
gucgaaucugcaugcagaaguuugcccgcaacgaccaccgcaccacccaua
ccaagauacacacgggcgagaagcccuuccagugucgaaucugcaugcaga
acuucagucagaaggcccaccugauccgccacauccgcacccacaccggcg
agaagccuuuugccugugacauuugugggaggaaauuugcccagaagggca
cccugggcgagcauaccaagauacacacgggaucucagaagcccuuccagu
gucgaaucugcaugcagaacuucagucgcggccgcgaccugucccgccaca
uccgcacccacaccggcgagaagccuuuugccugugacauuugugggagga
aauuugcccgccgcgacaaccugcacucccauaccaagauacaccugcggg
gaucccagcuggugaagagcgagcuggaggagaagaaguccgagcugcggc
acaagcugaaguacgugccccacgaguacaucgagcugaucgagaucgcca
ggaacagcacccaggaccgcauccuggagaugaaggugauggaguucuuca
ugaagguguacggcuacaggggaaagcaccugggcggaagcagaaagccug
acggcgccaucuauacagugggcagccccaucgauuacggcgugaucgugg
acacaaaggccuacagcggcggcuacaaucugccuaucggccaggccgacg
agaugcagagauacgugaaggagaaccagacccggaauaagcacaucaacc
ccaacgagugguggaagguguacccuagcagcgugaccgaguucaaguucc
uguucgugagcggccacuucagcggcaacuacaaggcccagcugaccaggc
ugaaccgcaaaaccaacugcaauggcgccgugcugagcguggaggagcugc
ugaucggcggcgagaugaucaaagccggcacccugacacuggaggaggugc
ggcgcaaguucaacaacggcgagaucaacuucugauaacucgagucuagaa
gcucgcuuucuugcuguccaauuucuauuaaagguuccuuuguucccuaag
uccaacuacuaaacugggggauauuaugaagggccuugagcaucuggauuc
ugccuaauaaaaaacauuuauuuucauugcugcgcuagaagcucgcuuucu
ugcuguccaauuucuauuaaagguuccuuuguucccuaaguccaacuacua
aacugggggauauuaugaagggccuugagcaucuggauucugccuaauaaa
aaacauuuauuuucauugcugcgggacauucuuaauuaaaaaaaaaaaaaa
aaaaaaaaaaaaaaaaaaaaaaaaaaaaaaaaaaaaaaaaaaaaaacuag.

### Example 2

### Cell Modification Method Development

In vitro studies: mobilized human CD34+ HSPCs were collected by apheresis from healthy subjects and purified. Purified HSPCs were transfected with ZFN mRNAs SB-mRENH1 and SBmRENH2. Untransfected CD34+ HSPCs from the same subjects served as controls. Forty-eight hours after transfection, the transfected CD34+ HSPCs (“ST-400”) were harvested and frozen for use in in vitro studies.

To analyze the effects of ZFN-mediated gene editing of the human. erythroid-specific enhancer of the BCL11A gene, the modified cells from above were placed in an in vitro erythropoiesis model known as “cRBC pooled differentiation” (Giarratana et al. (2011) Blood 118(19):5071), which entails culture for 21 days in a 3-step liquid culture with pro-erythroid cytokines. BCL11A enhancer gene modification was measured in ST-400 by MiSeq deep sequencing in DNA samples harvested 2 days after transfection, at the beginning of the in vitro differentiation and on Day 14 of the in vitro differentiation, prior to enucleation of a large fraction of the erythroid cells. Modification of the BCL11A enhancer locus in transfected cells included about 75% indels, within the range expected during production of clinical material. Gene modification levels were ≤0.2% in untransfected control HSPC.

Cell growth (expansion) was monitored over the course of the erythroid differentiation. Enucleation, a measure of erythroid maturation, was determined at Day 21. Expansion of transfected HSPCs ranged from about 2500- to 9000-fold and was approximately 2-fold lower than in untransfected HSPCs, reflecting the impact of the transfection procedure on early cell growth. The percent of enucleated cells did not differ between transfected and untransfected cells (ranging from about 59-62% in both cases).

Reverse-phase UPLC of protein samples isolated at Day 21 (the end point of the erythroid differentiation) was used to measure α-, β-, and γ-globin levels in the erythroid progeny of the transfected HSPCs.

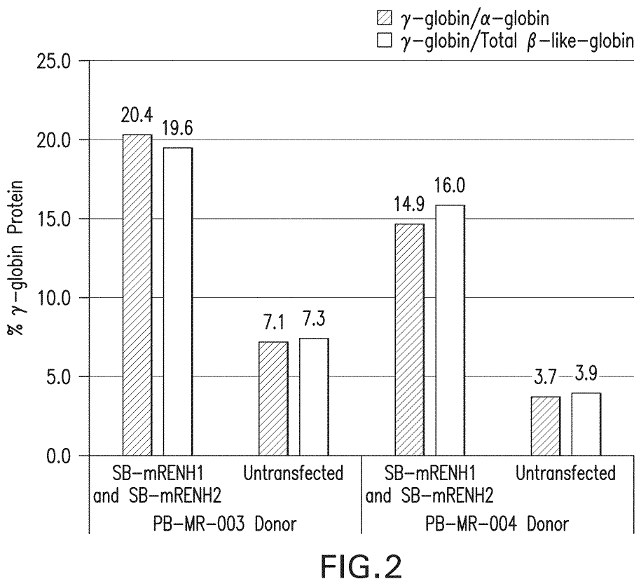

As shown in FIG. 2, the ratio of γ-globin to β-globin and of γ-globin to α-globin was increased approximately 3- to 4-fold in the erythroid progeny of ST-400 compared to the untransfected HSPCs. This finding demonstrates an outcome of that targeted gene modification results in elevation of γ-globin protein, which should increase HbF levels in the erythroid cells of patients with TDT. The observed increases in γ-globin levels are similar to those published for other methods targeting BCL11A (Wilber et al. (2011) Blood 117(10):2817-26) and those detected in patients with BCL11A haploinsufficiency (Basak et al. (2015) J Clin Invest 125(6):2363-8; Funnel et al. (2015) Blood 126(1):89-93).

For assessment of functional potential of the modified HSPC (assessed as proliferation and differentiation to hematopoietic lineages), the number and morphology of colonies formed by a fixed number of input cells in the CFU assay was used. Untransfected CD34+ HSPCs derived from the same subjects were used as negative controls. The CFU assay was performed using standard procedures. Briefly, triplicate cultures of 100 or 300 cells each were plated in 6-well plates, and incubated for 14 days, at which time point the cultures were scored for colony count and type of colony. Post-thaw viabilities were equivalent (about 72% to 83% in transfected HSPCs; about 96% in untransfected HSPCs). Percent plating efficacy of transfected HSPCs ranged from 15.7% to 453%, compared to 37.3% to 75.0% with untransfected HSPCs. The plating efficiency of ST-400 falls within ranges reported in other studies with gene-modified cells (Dever et al. (2016) Nature 539(7629)384-389; Wu et al. (2001) Gene Ther 8(5)384-90) and the lower efficiency compared to untransfected HSPCs is likely due to the impact of electroporation and gene modification.

As shown in Table 2, the modified HSPC differentiated into all hematopoietic lineages, including erythroid progenitors (CFU-E and BFU-E), granulocyte/macrophage progenitors (CFU-G/M/GM), and multi-potential progenitors (CFU-GEMM). The percentages of CFU-E derived from the modified HSPC were similar to those of the untransfected HSPCs, and the percentages of CFU-G/M/GM and CFU-GEMM were only minimally different. Thus, transfection and genetic modification with ZFN mRNAs SB-mRENH1 and SB-mRENH2 has minimal or no effect on the differentiation potential of the modified HSPC.

TABLE 2
Hematopoietic Differentiation of CD34+ HSPCs

| | Electroporation | | Average CFU | | | Average |
|---|---|---|---|---|---|---|
| Lot No. | condition | Cells/well | E | G/M/GM | GEMM | Total CFU |
| PB-MR-003 | mRENH1/mRENH2 | 100 | 16.3 | 17.7 | 0.0 | 34.0 |
| | untransfected | 100 | 25.3 | 45.3 | 4.3 | 75.0 |
| PB-MR-004 | mRENH1/mRENH2 | 100 | 10.0 | 35.7 | 0.0 | 45.7 |
| | untransfected | 100 | 10.7 | 45.0 | 1.0 | 56.7 |
| PB-MR-006 | mRENH1/mRENH2 | 300 | 17.7 | 29.3 | 0.6 | 47.7 |
| | untransfected | 100 | 20.7 | 32.0 | 1.0 | 53.7 |
| PB-CH-002 | mRENH1/mRENH2 | 300 | 12.7 | 38.3 | 0.0 | 51.0 |
| | untransfected | 100 | 9.0 | 27.0 | 1.3 | 37.3 |
| PB-CH-003 | mRENH1/mRENH2 | 300 | 17.3 | 28.7 | 1.0 | 47.0 |
| | untransfected | 100 | 19.7 | 40.3 | 0.6 | 60.7 |

Colony Formation in Soft Agar by ST-400-Transfected Fibroblasts: For assessment of transformation/tumorigenic potential, anchorage-independent growth of human WI-38 fibroblasts transfected with ZFN mRNAs SB-mRENH1- and SB-mRENH2 was assessed in a soft agar transformation assay. Gene modification levels were measured at 73% indels in the genetically modified WI-38 cells compared to ˜0.3% in untransfected WI-38 cells. No anchorage-independent growth of the transfected and untransfected WI-38 cells was observed at any time point. The results show that transfection with ZFN mRNAs SB-mRENH1 and SB-mRENH2, and the resultant ZFN-mediated disruption at the erythroid-specific enhancer of the BCL11A gene in WI-38 cells, did not promote tumorigenicity.

Karyotyping of modified CD34+HSPC: A karyotype analysis was conducted with the modified HSPCs. ZFNs are designed to induce DSBs in the genome at a specified target locus. Given their mechanism of action, it is possible that off-target activity of ZFNs could result in unplanned genetic changes. Visual examination of spread chromosomes from individual cells (karyotyping) can provide a global view of genetic integrity, and detect genetic abnormalities, including large-scale structural or numerical chromosomal changes that could be missed by other, more targeted tests.

To evaluate gross chromosomal morphologies, modified HSPC derived from 3 healthy subjects underwent karyotype analysis. Untransfected CD34+ HSPCs from the same subject served as control. MiSeq deep sequencing showed that gene modification at the erythroid-specific enhancer of the BCL11A gene was from 77% to 79% indels in the transfected HSPCs, compared to <0.1% indels in untransfected HSPCs. The karyotyping analysis showed that all cells were of human origin, and none had gross chromosomal abnormalities. Cytogenetic analyses of the modified HSPC showed no gross structural or numerical chromosomal abnormalities related to treatment.

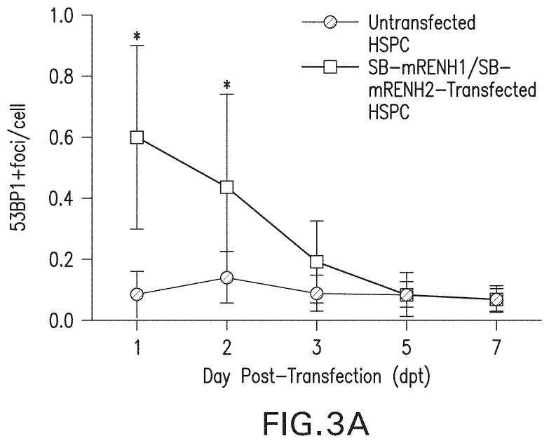

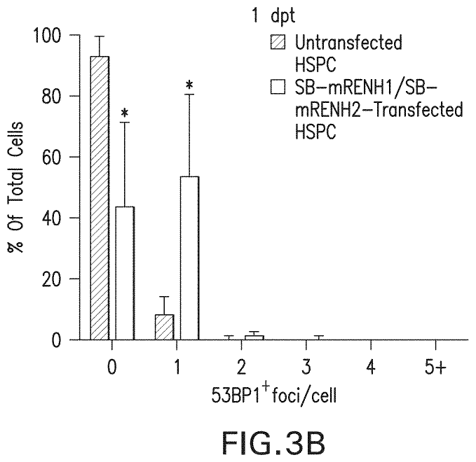

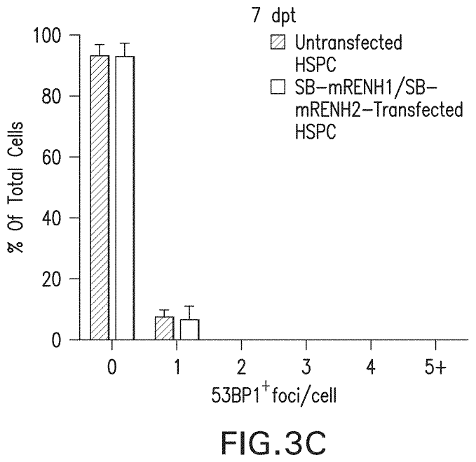

Double strand breaks in modified HSPC: modified HSPCs were tested in the p53-binding protein 1 (53BP1) assay to evaluate the duration and specificity of ZFN activity of over 7 days by immunohistochemistry. Gene modification levels were assessed on Days 1 and 2 post-transfection. 53BP1 is recruited to sites of DSBs within 24 hours after they occur and is involved in DSB repair via NHEJ, the major pathway for repair of ZFN-induced DSBs. The repair sites are visualized as intensely stained and distinct foci within the nucleus of fixed cells using immunofluorescence microscopy with antibodies to 53BP1. Assessment of DSBs using this method provides an unbiased temporal measure of net ZFN action (both on- and off-target). DSB in modified HSPCs were assessed, and results indicate that gene modification levels remained high as 53BP1 immunostaining levels decreased, demonstrating that the drop in 53BP1 signal was not due to loss of transfected cells over time (FIG. 3A through FIG. 3C). The highest levels of 53BP1 foci/cell were found 1-2 days post-transfection. In addition, about 50% of cells showed 1 53BP1 foci/cell (1 DSB/cell) 1-day post-transfection and about 8% of cells 7 days post-transfection (similar to background levels seen with untransfected cells). Gene modification at the BCL11A enhancer target were 73.5% and 78.5% on Days 1 and 2 post-transfection.

Translocation assay in modified HSPCs. A molecular translocation assay was conducted with the modified HSPCs to evaluate potential translocation events. The frequency of translocation events occurring between the on-target locus (BCL11A enhancer) and all known sites of off-target cleavage (19 identified previously) were quantified. Twelve of these were identified via a standard, MiSeq-based deep sequencing of candidate off-target sites, and yielded indel levels in the ˜0.01% to ˜0.1% range. The remaining seven sites were identified via a more intensive assessment of a smaller locus panel via oversampling with ultra-deep sequencing NextSeq platform, and yielded indel rates in the lower range of ˜0.001% to ˜0.01%. This is a highly sensitive approach and enables detection of translocation events at frequencies approaching one in 105 queried genomes; it was used to query for the presence and level of reciprocal translocations between the intended cleavage target in the BCL11A erythroid enhancer and each previously identified off-target site.

Thus, ST-400 ZFN pair is highly specific for the erythroid-specific enhancer of the BCL11A gene and has but a minimal amount of detectable off-target activity. In particular, MiSeq deep DNA sequencing showed very low levels of off-target cleavage of 0.15% or less and NextSeq analysis revealed extremely low levels of off-target cleavage of less than 0.01%. In comparison, indel levels at the targeted erythroid-specific enhancer of the BCL11A gene ranged from about 79 to 86%. These genome wide analyses indicate that indel levels at the BCL11A on-target locus exceed the levels of modification at all identified off-target sites combined by more than a factor of 300. Furthermore, bioinformatics analysis in conjunction with a literature review of identified off-target loci showed no evidence of modifications to coding regions of genes involved in critical hematopoietic functions and off-target events did not lead to modifications that are known to be associated with hematopoietic malignancies in humans.

Off-target Transcriptional Effects by the ST-400 ZFN Pair in Erythroid Progeny: To assess off target transcriptional activity of the optimized ST-400 ZFN pair, the expression profile of 11 genes flanking the BCLA11A gene were analyzed using MiSeq deep sequencing. RNA was collected from the transfected CD34+ HSPCs on Day 14, at which time gene modification levels at the erythroid-specific enhancer of BCL11A gene were quantitated as >50% compared to control. Levels of γ-globin mRNA in the transfected CD34+ HSPCs were increased about 2-fold (normalized to 18s RNA), reflecting decreased BCL11A expression resulting from the on-target elimination of the GATA1 binding site in the erythroid-specific enhancer of the BCL11A gene. In contrast, the expression levels of the 11 genes flanking the BCL11A gene were similar to those of the 11 genes in the control cells. Expression levels of 4 other genes regulated by GATA1 (KLF1, SCL4A1, ZFPM1 and ALAS2) also were not affected. These results show that the activity of ZFN mRNAs SB-mRENH1 and SB-mRENH2 is restricted to repression of BCL11A gene transcription and its consequent downstream effects.

The method used to detect translocation events was an adaptation of a standard TaqMan assay for DNA quantitation (Holland et al. (1991) Proc Natl Acad Sci U.S.A. 8(16):7276-7280) in which polymerase chain reaction (PCR) is performed in conjunction with a probe that releases a fluorophore upon annealing to DNA and subsequent degradation by the DNA polymerase. In the intact probe the fluorophore signal is suppressed via interaction with covalently attached quenchers. The probe is designed to anneal inside the region that is being amplified by the PCR primers. The fluorescent signal detected is thus proportional to the amount of amplicon present in the sample. TaqMan primers were designed to be 20 bases long and yield amplicons that span approximately 200 base pairs (bp). Primers were synthesized and purified using standard desalting. Fluorescent probes were designed to span 20 bp and have 60% GC content. The probes contained a 5′ HEX reporter dye, a 3′ Iowa Black FQ quencher, and an additional internal “ZEN” quencher to further reduce background signal. Probes were HPLC purified.

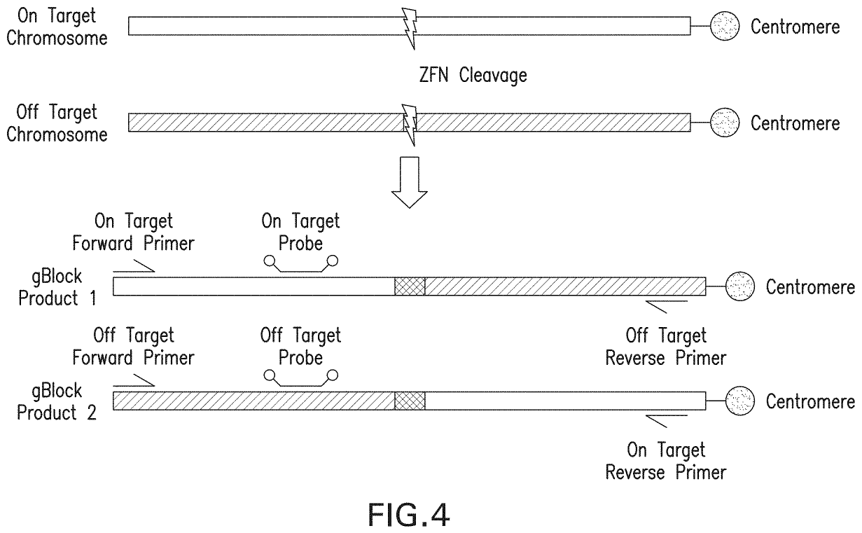

As the queried translocation sequences are not found in the native human genome, synthetic DNA fragments encompassing the predicted translocation junctions needed to be designed for each of the 19 off-target loci in order to generate standard curves and assess assay sensitivity. A schematic depiction of these reagents, along with corresponding primer and probe reagents is provided in in FIG. 4. Note that a unique 21 bp sequence was inserted within each positive control template between the BCL11A and off-target derived segments to enable sequencing-based discernment from bona fide translocation products, in the event of suspected contamination. Synthetic double-stranded DNA fragments were purchased as gBlocks where the lengths of the DNA fragments ranged from 287 to 434 bp. In FIG. 4, the top panel depicts chromosome segments encompassing the BCL11A enhancer on-target site (green) and an off -target site (orange). The bottom panel sketches positive control reagents (gBlocks) for detection of the corresponding translocation products. Also shown are the approximate primer and probe locations used in the TaqMan assay. The maroon segment within each gBlock is a unique sequence inserted into each control reagent to distinguish it from a true translocation product and allow for monitoring of potential cross-contamination. Product gBlocks were probed in the BCL11A region of the fragment. Product 2 gBlocks were probed in the off-target region of the fragment.

For each queried translocation (Product 1 or Product 2, see, FIG. 4), a standard curve was generated that contained 10000, 1000, 100, 10, 3, 1, 0.1, or 0.01 copies of synthetic gBlock DNA in the context of 100,000 haploid genomes of CD34+ genomic DNA (gDNA) from untransfected cells. The two lowest points on the standard curve (0,1 and 0.01 copies) were expected to yield negative signals and were generated to provide additional verification of control DNA quantitation and assay robustness. The three-copy point was included to provide higher resolution and additional data points in the range of the expected limit of detection for this assay. Ten-fold dilutions were made in AE Buffer (10 mM Tris-Cl and 0.5 mM EDTA pH 9.0) from the QIAGEN DNeasy Blood and Tissue Kit (QIAGEN) containing 5 ng/uL of gDNA from K562 cells as carrier before transferring into tubes containing 100,000 genomes of CD34+ gDNA.

Reactions were prepared using the Bio-Rad ddPCR 2× Supermix (Bio-Rad; Hercules, Calif.) that contained PCR buffer, dNTPs, and DNA polymerase as per the manufacturer's protocol. DNA templates for the standard curves (gBlocks) were generated. The NTC (no template control) sample lacked added gBlock but did include 330 ng of gDNA from untransfected CD34+ cells. Each reaction contained 0.5 μM primers and 0.25 μM probe. Genomic DNA from each of the three lots of ST-400 was purified using the QIAGEN DNeasy Blood and Tissue Kit. For each tested sample, 100,000 haploid genomes (330 ng) of DNA from the indicated lot was added to each reaction to match the conditions used to generate the standard curve. All samples and standards were run in triplicate. The TaqMan assay was run on a Bio-Rad CFX 96 Real-Time PCR Detection System as per the manufacturer's instructions. The PCR program used was as follows: 95° C. for 10 min followed by 50 cycles of 94° C. for 30 sec and 59° C. for one min.

A TaqMan assay was performed to examine genome DNA from ZFN-treated CD34+ cells for evidence of translocations between the BCL11A on-target site and the 12 off-target loci that had been identified via MiSeq analysis. To accomplish this, CD34+ cells from mobilized peripheral blood were treated with ST-400 ZFNs using clinical conditions for RNA transfection and expression. After two days, gDNA was isolated and submitted for assessment of reciprocal translocations (Product 1 and Product 2). The results, which are summarized in Table 3, revealed very low translocation signals for seven of the off-target sites, with frequencies in the range of one translocation for every 10⁴ to 10⁶ haploid genomes. The remaining sites showed no evidence of translocations.

TABLE 3
Translocations detected

| | | | Average | | | | | | | | |
|---|---|---|---|---|---|---|---|---|---|---|---|
| | | | translocation | | Translocations per 100,000 haploid | | | | | | |
| | | | level | | genomes, by CMC lot | | | | | | |
| | | | (per 100,000 | | Product 1 | | | Product 2 | | | |
| Off-target site | | | haploid genomes) | | PB- | PB- | PB- | PB- | PB- | PB- | |
| Name | | | Product | Product | MR- | CH- | CH- | MR- | CH- | CH- | |
| (OT#) | Location | | 1 | 2 | 006 | 001 | 002 | 006 | 001 | 002 | Locus ID |
| OT1 | chr8 | 119856440 | 2.22 | 0.99 | 2.17 | 0.67 | 3.83 | 1. | 1.47 | 0.5 | NIFMAEVG |
| OT10 | chr2 | 23702834 | 3.59 | 7.01 | 6.73 | 2.07 | 1.97 | 3.57 | 8.43 | 9.03 | RFGIYSHZ |
| OT3 | chr6 | 89888012 | 0.14 | 0.31 | 0. | 0.42 | 0. | 0.43 | 0. | 0.5 | FYQYHJIS |
| OT6 | chr10 | 132654832 | 0.4 | 1.49 | 0. | 0. | 1.2 | 1.67 | 0. | 2.8 | ALVTVCYL |
| OT2 | chr1 | 21635648 | 0.12 | 0. | 0 | 0. | 0.37 | 0. | 0. | 0. | PEBPWNNJ |
| OT4 | chrX | 66004390 | 0.08 | 0. | 0. | 0.23 | 0. | 0. | 0. | 0. | MKRBBTRS |
| OT12 | chr19 | 51327822 | 0. | 0.39 | 0 | 0. | 0. | 1.17 | 0. | 0. | YJJYYWPK |
| OT8 | chr8 | 94988044 | 0. | 0. | 0. | 0. | 0. | 0. | 0. | 0. | CSRBEMTR |
| OT7 | chr7 | 131503656 | 0. | 0. | 0. | 0. | 0. | 0. | 0. | 0. | LSKZRNJH |
| OT9 | chr20 | 37707466 | 0. | 0. | 0. | 0. | 0. | 0. | 0. | 0. | MDRSNDIS |
| OT11 | chr16 | 2122340 | 0. | 0. | 0. | 0. | 0. | 0. | 0. | 0. | FYTLXRTA |
| OT5 | chr3 | 49724756 | 0. | 0. | 0. | 0. | 0. | 0. | 0. | 0. | TNJMZKZS |

### Example 3

### Clinical Study of Modified HSPCs

A study was undertaken in humans to test the safety of using modified HSPCs to treat TDT. In addition, assessment of the efficacy of the modified HSPCs was evaluated. Exploratory objective also included evaluating the gene modification characteristics (% and durability) at the erythroid-specific enhancer of the BCL11A gene after treatment with the modified cells and assessment of the impact of the modified cells on the biochemical, imaging, functional, and bone marrow evaluations related to β-thalassemia and HSCT.

Inclusion criteria for the study included six subjects (β⁰/β⁰ or non-β⁰/β⁰) between the ages of 18 and 40 years old with a clinical diagnosis of TDT with ≤8 documented PRBC transfusion events per year on an annualized basis in the two years prior to screening for the study. Also required was a confirmed molecular genetic diagnosis of β-thalassemia. Subjects included males and females willing to use birth control.

Key exclusion criteria for subjects in the study included: previous history of autologous or allogenic human stern cell transplant or solid organ transplant; γ-globin allelic variants associated with clinically significant altered oxygen affinity (examples include, but are not limited to, Hb F-Poole, Hb F-M Osaka, Hb F-La Grange, Hb F-Cincinnati and large deletions such as γβ-thalassemia or εγδβ-thalassemia); medical contraindication to apheresis; massive splenomegaly and Absolute neutrophil count (ANC) ≤1,000/μL; renal dysfunction as defined by serum creatinine ≥2.0 mg/dL; bridging fibrosis, liver cirrhosis, or active hepatitis based on liver biopsy obtained in previous 12 months or at screening; treatment with prohibited medications in previous 30 days; clinically significant active bacterial, viral, fungal, or parasitic infection; diagnosis of HIV or evidence of active HBV or HCV infection based on screening laboratory testing; Karnofsky performance scale ≤60; corrected DLCO ≤50% of predicted or clinically-significant restrictive; lung disease based on Screening pulmonary function tests (PFTs); congestive heart failure (NYHA Class III or IV); unstable angina, uncontrolled arrhythmia, or left ventricular ejection fraction (LVED) <40%, QTcF >500 msec based on Screening ECG, cardiac T2*MRI <10 msec based on Screening MRI; history of significant bleeding disorder; current diagnosis of uncontrolled seizures; history of active malignancy in past 5 years (non-melanoma skin cancer or cervical cancer in situ permitted); any history of hematologic malignancy, or family history of cancer predisposition syndrome without negative testing result in the study candidate; history of or active alcohol or substance abuse that may interfere with study compliance; history of therapeutic non-adherence; currently participating in another clinical trial using an investigational study medication, or participation in such a trial within 90 days or less than 5 half-lives of the investigational product prior to Screening visit; previous treatment with gene therapy; allergy or hypersensitivity to busulfan or study drug excipients (human serum albumin, DMSO, and Dextran 40); or any other reason that would render the subject unsuitable for participation in the study.

Study Design

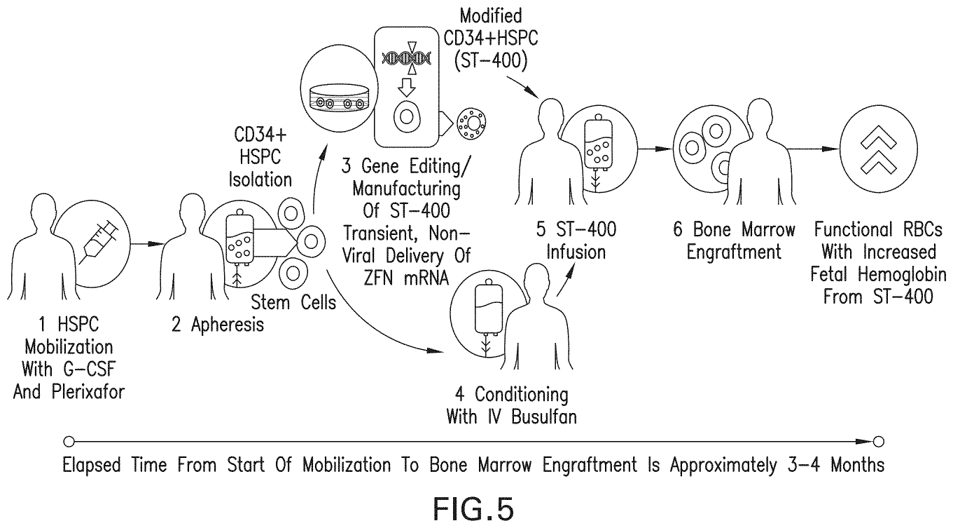

The study was performed on subjects with transfusion-dependent β-thalassemia (TDT). Upon enrollment, eligible subjects undergo apheresis to collect autologous CD34+ HSPCs. The CD34+ HSPCs were treated ex vivo by transfection with ZFN mRNAs SB-mRENH1 and SB-mRENH2 to manufacture the study drug. Subjects receive conditioning therapy with intravenous (IV) busulfan before being infused with the modified HSPCs. CD34+ HSPCs were mobilized in each subject using treatment with G-CSIF and plerixafor. Mobilized CD34+ HSPCs were collected from each subject on Days 5 and 6 (+/−Day 7 if needed to secure the rescue treatment) of mobilization by apheresis. CD34+ HSPCs were mobilized in each subject following G-CSF (on Days 1-6 of mobilization) and plerixafor (on Days 4, 5, and 6 of mobilization) administration (see, FIG. 5). Mobilized CD34+ HSPCs were collected from each subject by apheresis on two consecutive days (e.g., Days 5 and 6) and unmanipulated back-up grafts were collected on the third day (e.g., Day 7 to secure the rescue treatment) with a target of 25×10⁶ CD34+ HSPCs/kg total, although smaller yields are acceptable. If needed, a second mobilization and apheresis cycle was performed ≥2 weeks later.

The collected cells of each subject were split into 2 portions, one portion for modified HSPC drug manufacturing and the other portion set aside in the event a rescue treatment is indicated.

The rescue treatment portion comprises a minimum of 2.5×106 CD34+ HSPCs/kg. The rescue treatment portion was cryopreserved unmodified and stored at the study site for availability in the event of delayed hematopoietic reconstitution or graft failure with aplasia. If the first apheresis cycle did not mobilize the minimum number of CD34+ HSPCs required for modified HSPC drug manufacturing and for rescue treatment, the mobilization procedure may be repeated. Selection of the timing of a second apheresis was at the discretion of the Investigator based on the subject's clinical status, but was no sooner than 2 weeks (≥2 weeks) after the initial apheresis.

After removal and storage of the rescue treatment, the remainder of the subject's mobilized and harvested cells were sent by courier to the GMP manufacturing facility. A CD34+ cell selection followed by transfection with ZFN mRNAs SB-mRENH1 and SB-mRENH2 to disrupt the erythroid-specific enhancer of the BCL11A gene was performed to generate the modified HSPC study drug. The modified HSPC were cryopreserved and stored until all the clinical protocol segments up to and including the Baseline visit procedures are completed and the subject is ready for infusion. The modified HSPC were cryopreserved in 50 mL CryoMACS® freezing bags (fill volume of approximately 10 to 20 mL; total cell count of approximately 1.0×108 to 2.0×108 cells) using a controlled rate freezer. Multiple freezing bags were used if cell yield exceeds the capacity of a single bag. Infusion bags were stored in vapor phase liquid nitrogen (at <−150 oC) at the manufacturing facility until they ready to be shipped to the clinical study center.

After release of the modified HSPC for clinical use, subjects were admitted to the hospital to begin IV busulfan in a dedicated transplant unit. Subjects received a myeloablative regimen of busulfan (3.2 mg/kg/day; IV via central venous catheter) for 4 days (total dose of 12.8 mg/kg, which is considered standard-of-care for autologous transplantation) on Days −6 through −3 before infusion of the modified HSPC on Day 0. IV busulfan may be dosed once daily (total of 4 doses) or every 6 hours (total of 16 doses) according to study center practices or guidelines. After the first dose, the IV busulfan dose was adjusted based on pharmacokinetic sampling and study center practices to target an area under the curve (AUC) of 4,000-5,000 mmol*min for daily dosing or an AUC of 1,000-1,250 mmol*min for every 6 hour dosing for a total regimen target AUC of 16,000-20,000 mmol*min. IV busulfan pharmacokinetic targeting may be modified for subsequent subjects based on experience with previous subjects after discussion with the Safety Monitoring Committee (SMC). Therapeutic drug monitoring to determine clearance of busulfan after 4 days of dosing was not required but may be performed at the discretion of the Investigator in accordance with study center practices (see, FIG. 5).

Modified HSPC infusion: After myeloablative conditioning with intravenous busulfan (total regimen targeted exposure=16,000 to 20,000 μmol*min as confirmed and/or adjusted based on pharmacokinetic sampling), patients received the thawed CD34+ HSPCs (“ST-400”) product by central venous catheter infusion (FIG. 5). The frozen modified HSPC were thawed and infused, such that the entire process of thawing and infusion is finished within about 15 minutes. The volume of frozen modified HSPC was determined by the subject's weight. Vital signs (blood pressure, temperature, heart rate, respiratory rate and pulse oximetry) were monitored prior to infusion and afterwards.

Once given the study drug, the subjects were monitored for routine lab work. In addition, assessment of any adverse events will be done, and blood cells were assayed for gene modification. HbF levels will also be evaluated, endocrine function analyzed, and MRIs performed to assess iron load. Kinetics and success of hematopoietic reconstitution, duration of hospitalization after conditioning, screening for potential development of hematological malignancies, quality-of-life by Short Point Health Status Survey (SF-36 Survey), overall function by Karnofsky performance score, efficiency of apheresis procedure, difference between % indels in ST-400 product and indels detected in hone marrow and blood following ST-400 infusion will be evaluated.

An AE is any untoward medical occurrence associated with the use of a drug in humans, whether or not considered drug-related. An AE can include any of the following events that develop or increase in severity during this study: any sign, symptom, or physical examination finding that worsens in nature, severity, or frequency compared to baseline status (i.e., prior to screening), whether thought to be related or unrelated to the condition under study, any clinically significant laboratory abnormality or laboratory abnormality that requires medication or hospitalization. Abnormal laboratory results will be graded based on Common Terminology Criteria for Adverse Events (CTCAE) 5.0 criteria, a Grade 1 or 2 clinical laboratory abnormality should be reported as an AE only if it is considered clinically significant by the Investigator, a Grade 3 and 4 clinical laboratory abnormality that represents an increase in severity from baseline should be reported as an AE if it is not associated with a diagnosis already reported on the CRF, all events associated with the use of treatment, including those occurring as a result of an overdose, abuse, withdrawal phenomena, sensitivity, or toxicity to the treatment, concurrent illness, injury or accident.

A SAE is any AE that results in any of the following outcomes: death, life-threatening threatening event (i.e., an event that places the subject at immediate risk of death); however, this does not include an event that, had it occurred in a more severe form, might have caused death, inpatient hospitalization or prolongation of existing hospitalization, persistent or significant incapacity or substantial disruption of the ability to conduct normal life functions, congenital anomaly/birth defect in the offspring of an exposed subject, or a medically important event.

Evaluation of secondary and exploratory events: Baseline levels of HbF fractions (A and F in g/dL) and percent HbF will be determined based on the last assessment on or prior to the date of first administration of IV busulfan. HbF levels and change from baseline will be summarized by study visit.

Baseline frequency and volume of PRBC transfusions are based on the 2-year period prior to Screening. Frequency and volume of transfusion is annualized by study period and overall, and compared descriptively to the baseline values.

Monitoring modified HSPC heterogenicity following infusion: Following infusion, the modified HSPC may be monitored in the patient to determine engraftment efficiency and modification heterogenicity as assessed by indel profile. Subject cell samples may be purified from the peripheral blood, bone marrow aspirate or other tissue samples (about 5×104 to 1×107 cells preferably) and subject to genomic DNA isolation. The region around the cleavage site is then amplified by PCR under standard conditions. A second round of PCR is then performed to add adapters such that the reaction may be analyzed using MiSeq (Illumina). The sequencing data from the subject cells is compared with a standard curve to determine percent indels.

The protocol directed that patients 2 and 3 could not begin chemotherapy conditioning until the previous patient demonstrated neutrophil and platelet engraftment; following successful engraftment of patient 3, patients 4-6 could begin chemotherapy conditioning. Patients were monitored for safety and efficacy. The study encompasses follow-up for 3 years, after which patients are offered participation in a long-term safety follow-up study.

Safety and tolerability were assessed by incidence of adverse events (AEs) and serious AEs (SAEs). Success and kinetics of hematopoietic reconstitution were assessed by neutrophil (ANC ≥500 cells/μL) and platelet (≥20,000 cells/μL unsupported by transfusion) engraftment. On-target indel patterns were tracked at the molecular level over time for surveillance of emerging hematopoietic clones. Patients were monitored for presence of on-target indels in hematopoietic cells, fetal hemoglobin levels, and transfusion requirements following ST-400 infusion; post-transplantation hemoglobin transfusion thresholds were per clinical sites' standard practice (Patients 1 and 2: <8 g/dL; Patient 3: <7 g/dL).

Results

To date, autologous ST-400 product has been manufactured for 5 of the 6 patients, 3 of whom have received ST-400 (Table A). Safety and efficacy data; adverse events, fetal hemoglobin production, indel markings and PRBC transfusion requirements for these patients will evolve over time, potentially for 12 months or longer.

Table A
Patient Demographics and Disease Characteristics

| | | | Annualized | |
|---|---|---|---|---|
| | Age at | | PRBC | Time |
| | Consent | | Events Pre- | Post |
| Patient | (Years) | Genotype | Enrollment | Infusion |
| 1 | 36 | β⁰ | 27 | 39 |
| | | β⁰ | | Weeks |
| 2 | 30 | β⁺ (severe IVS-I-5: | 18 | 26 |
| | | G > C ) | | Weeks |
| | | β⁺ (severe IVS-I-5: | | |
| | | G > C) | | |
| 3 | 23 | β⁰ | 15 | 12 |
| | | β⁺ (severe IVS-II-654 | | Weeks |
| | | C > T) | | |
| 4 | 18 | β^{WT} (αα) | 13 | Pre- |
| | | β⁰ (αααα) | | Infusion |
| 5 | 35 | β⁰ | 15 | Pre- |
| | | β⁺ (severe IVS-I-110 | | Infusion |
| | | G > A) | | |

β⁰, absence of β-globin production;
β⁺, decreased β-globin production;
β^{WT}, wild type (normal β-globin production);
PRBC, packed red blood cell transfusion.

The first patient (Patient 1) treated with ST-400 in the Phase 1/2 study has the most severe form of transfusion-dependent beta thalassemia (β0/β0). Over the two years prior to treatment in the study, this patient received packed red blood cell (PRBC) transfusions every-other-week. During the ST-400 infusion, Patient 1 experienced a transient allergic reaction considered related to the cryoprotectant present in the product. Thereafter, the post-transplant clinical course was routine, and the patient demonstrated neutrophil and platelet recovery within two and four weeks of infusion, respectively.

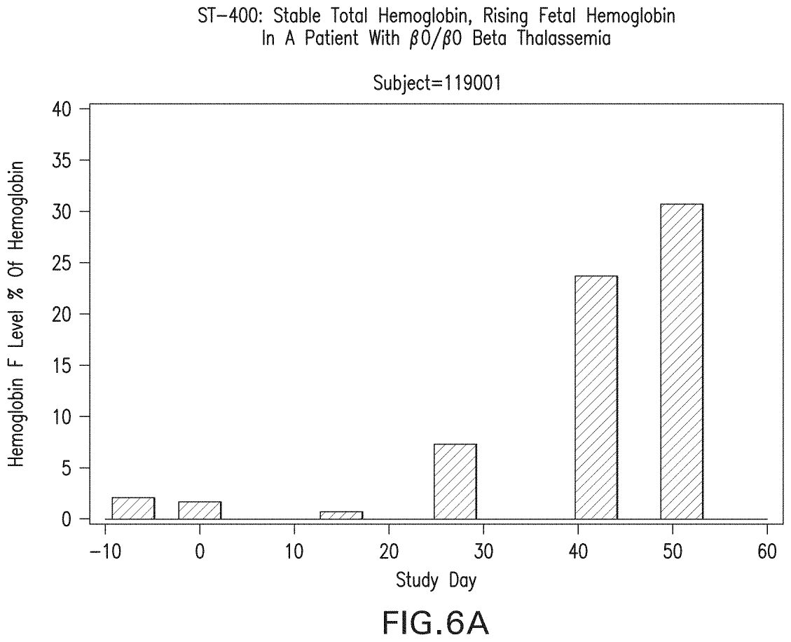

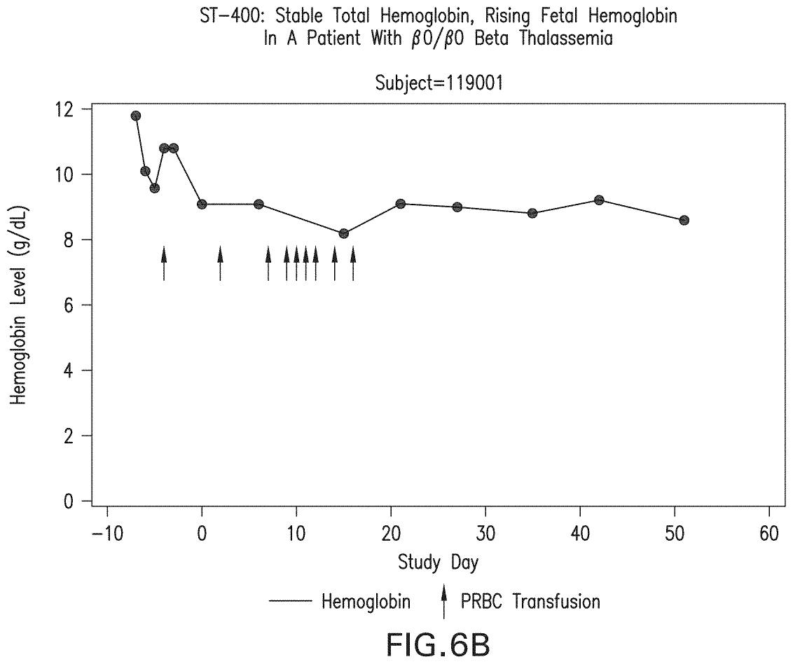

Patient 1 received a PRBC transfusion two weeks after the modified HSPC infusion and did not require further PRBC infusions during the following 6 weeks. At seven weeks post infusion with the modified HSPC, total hemoglobin levels remained stable at about 9 and levels of fetal hemoglobin continue to rise (from approximately 1% of total hemoglobin at time of infusion to 31% (see, FIG. 6A and FIG. 6B). Indels (insertions or deletions that are created at the targeted sequence of DNA) have been detected in circulating white blood cells, indicating successful editing of the BCL11A gene and disruption of the BCL11A erythroid specific enhancer, which is intended to upregulate endogenous fetal hemoglobin production in red blood cells.

Following demonstrated neutrophil and platelet engraftment in patient 1, patients 2 and 3 were also treated as described above. Patients 1, 2 and 3 all have severe beta thalassemia genotypes: β0/β0, homozygous for the severe β+ IVS-I-5 (G>C) mutation (Patients 1 and 3) or β0/β+ genotype including the severe IVS-II-654 (C>T) mutation (Patient 2).

Patient 1 and Patient 2 experienced prompt hematopoietic reconstitution. Patient 1 had increasing fetal hemoglobin (HbF) fraction that contributed to stable total hemoglobin. After being free from PRBC transfusions for a total of 6 weeks, Patient 1 subsequently required intermittent transfusions. Patient 2 had rising HbF levels observed through 90 days post-infusion. For Patients 1 and 2, on-target insertions and deletions (indels) were present in circulating white blood cells. Patient 3 had just completed ST-400 manufacturing and HbF levels will be determined after infusion.

Patient 1 experienced a serious adverse event (SAE) of hypersensitivity during ST-400 infusion considered to be related to the product by the investigator. This event was resolved with treatment. No other SAEs related to ST-400 have been reported. No clonal hematopoiesis has been observed.

Regular follow up (evaluating hematopoietic reconstitution, fetal hemoglobin levels, indels in circulating white blood cells, etc.) is conducted over time (e.g., 12 or more months) as the modified stem cells repopulate the marrow and drive hematopoiesis, HbF levels are increased and the need for transfusions reduced or eliminated in the patients.

Mobilization and Apheresis Outcomes

Peripheral blood CD34+ counts before daily apheresis varied from 25 to 118 cells/μL. Patient 1 underwent 2 cycles of mobilization and apheresis due to low cell dose and CFU potency in the first ST-400 lot. The back-up graft was cryopreserved from the first cycle. Patients 2, 3, 4 and 5 each underwent one cycle of mobilization and apheresis from which their ST-400 lots were manufactured, and back-up grafts cryopreserved.

Product Characteristics and Hematopoietic Reconstitution

On-target indels in the ST-400 product ranged from 23-80% as shown below in Table B.

TABLE B
ST-400 Product Characteristics and Hematopoietic Reconstitution

| | Cell | | CFU | On- | | Platelet |
|---|---|---|---|---|---|---|
| | Dose | | Dose | target | Neutrophil | Engraft- |
| | (10⁶/ | CD34+ | (10⁵/ | Indels^{a} | Engraftment^{b} | ment^{c} |
| Patient | kg) | (%) | kg) | (%) | Day (s) | Day (s) |
| 1^{d} | 5.9 | 91 | 6.2 | 23^{e} | 14 | 25 |
| 2^{d} | 4.5 | 87 | 4.0 | 73 | 15 | 22 |
| 3^{f} | 11.4 | 90 | 14.8 | 54 | 22 | 35 |
| 4 | 5.4 | 86 | 7.6 | 80 | Pre-Infusion | Pre-Infusion |
| 5 | 9.5 | 98 | 10.5 | 76 | Pre-Infusion | Pre-Infusion |

^{a}Percentage of all BCL11A ESE alleles with an indel; this is not equivalent to the percent of all cells with at least one edited BCL11A ESE allele.
^{b}Neutrophil engraftment defined as occurring on the first of 3 consecutive days on which the patient's neutrophil count was ≥500 cells/μL.
^{c}Platelet engraftment defined as occurring on the first of 3 consecutive measurements spanning a minimum of 3 days (in the absence of platelet transfusion in the preceding 7 days) on which the patient's platelet count was ≥20,000 cells/μL.
^{d}Patients 1 and 2 received G-CSF from day +5 through neutrophil engraftment per site's standard operating procedure.
^{e}Patient 1 underwent 2 cycles of apheresis and manufacturing of ST-400; on-target indel percentage for the lot not shown was 26%. All other patients underwent only one cycle of apheresis and manufacturing,
^{f}Patient 3 received G-CSF from day +21 through neutrophil engraftment per site's standard operating procedure.

As shown, the lowest indel value was seen in Patient 1, for whom editing efficiency was near 25% in two separately manufactured lots. Using the same manufacturing process at clinical scale, CD34+ cells from 12 healthy donors were efficiently edited: median on-target indels, 71%; range, 59% to 83%. ST-400 viable nucleated cell doses were 4.5-11×10⁶ cells/kg. Patients demonstrated neutrophil engraftment in 14-22 days and platelet engraftment in 22-35 days.

Safety

No emerging clonal hematopoiesis has been observed by on-target indel pattern monitoring over time by indel profiling in the 3 dosed patients, See, FIG. 7A through FIG. 7C.

Through observations in Patient 1 at month 9; patient 2 at month 6) and Patient 3 at day 56, the maximum frequency of a unique indel at any timepoint has been 16%, 16% and 14% of all indels detected, respectively.

Reported serious adverse events (SAE) are shown in Table C for treated patients.

TABLE C
Serious Adverse Events

| | Serious Adverse | Related |
|---|---|---|
| Patient | Events | to ST-400 |
| 1 | Hypersensitivity^{a} | RELATED |
| 2 | None | — |
| 3 | Pneumonia^{b} | NOT |
| | | RELATED |
| 4 | None | — |
| 5 | None | — |

^{a}Occurred during infusion of ST-400 and rapidly resolved with medical management; considered related to DMSO cryoprotectant.
^{b}Pneumonia occurred in the time period between the apheresis procedure and the start of chemotherapy conditioning.

As shown, only one SAE attributed to ST-400 drug product was reported; this SAE of hypersensitivity occurred during ST-400 infusion, resolved by the end of infusion, and was considered related to the product cryoprotectant, DMSO.

Otherwise, reported AEs have been consistent with the known toxicities of mobilization, apheresis and myeloablative busulfan conditioning.

Changes Following Infusion

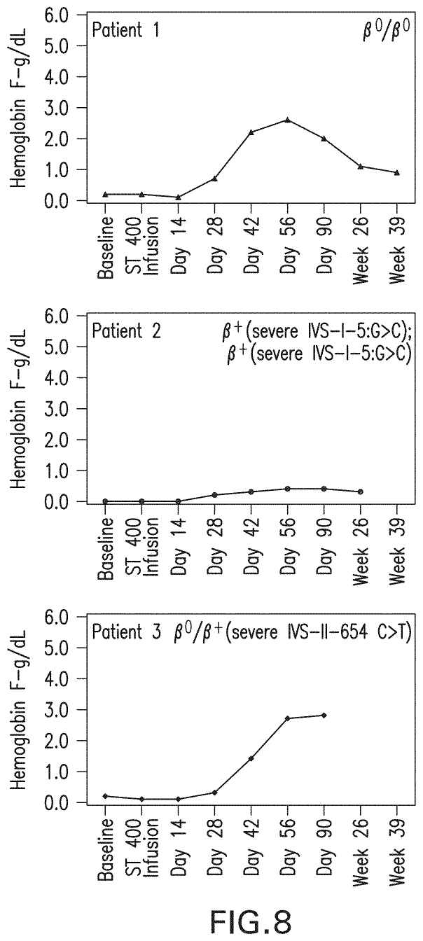

As described above, following ST-400 transplantation, fetal hemoglobin levels increased compared with baseline in all 3 patients (FIG. 8), with Patients 1 and 3 showing greater induction than Patient 2, consistent with the fact that Patient 2 received ST-400 product with the lowest cell dose and CFU potency.

In Patient 1, indels have persisted in peripheral leukocytes through month 9, and day 90 unfractionated marrow cells showed 6% indels. After an initial transfusion-free period of 6 weeks, this patient has resumed intermittent PRBC transfusions, with a 33% reduction in projected annualized PRBC units transfused at approximately 8 months since engraftment.

In Patient 2, indels have persisted in peripheral leukocytes through month 6, and day 90 unfractionated marrow cells showed 32% indels. The patient is receiving intermittent PRBC transfusions.

In Patient 3, indels have persisted in peripheral leukocytes through day 56. Assessment of a marrow aspirate sample at 90 days is not yet available. Following an initial transfusion-free period of 7 weeks, the patient has received two PRBC transfusions beginning at 62 days post-infusion.

Summary of Patients 1 to 3

Treatment of and results for patients 1 to 3 are summarized below. For each patient, CD34+ cell dose was calculated as follows: CD34+ dose=[total cell dose]×[CD34+%]. See, e.g., Table B, showing total cell dose in column 2 and CD34+% in column 3.

### Patient 1

Patient 1 has a β0/β0 genotype, the most severe form of TDT, and had 27 annualized packed red blood cell (PRBC) events prior to enrollment into the study. The patient underwent a second cycle of mobilization and apheresis due to the low cell dose and potency achieved in the first cycle. In both ST-400 lots, editing efficiency was approximately 25%, which was lower than the other patients enrolled in the study and 12 trial-run lots manufactured at clinical scale (71% median editing efficiency).

On-target indels in the infused ST-400 product were 23%, and the CD34+ cell dose was 5.4×106 cells/kg. Indels were present in unfractionated marrow cells at 90 days and have persisted in peripheral leukocytes through Month 9. Following ST-400 infusion, fetal hemoglobin levels increased to approximately 2.7 g/dL at Day 56 and remained elevated compared to baseline at 0.9 g/dL at week 39, the most recent measurement at the time of the ASH data cut. After an initial transfusion-free duration of 6 weeks, the patient resumed intermittent PRBC transfusions, with an overall 33% reduction in annualized PRBC units transfused since engraftment.

### Patient 2

Patient 2 is homozygous for the severe β+ IVS-I-5 (G>C) mutation and had 18 annualized PRBC events prior to enrollment into the study. On-target indels in the ST-400 product were 73%, with a CD34+ cell dose of 3.9×10⁶ cells/kg, the lowest seen across the ST-400 lots manufactured for the 5 enrolled patients. Indels were present in unfractionated marrow cells at 90 days and have persisted in peripheral leukocytes through Month 6. Following ST-400 infusion, fetal hemoglobin levels increased as compared with baseline, but have been <1 g/dL through to 26 weeks, the lowest induction level observed in the three patients treated to date. The patient is currently receiving intermittent PRBC transfusions.

### Patient 3

Patient 3 has a β0/β+ genotype that includes the severe IVS-II-654 (C>T) mutation and had 15 annualized PRBC events prior to enrollment into the study. On-target indels in the ST-400 product were 54%, with a CD34+ cell dose of 10.3×10⁶ cells/kg. At the time of the ASH data cut indels have persisted in peripheral leukocytes through Day 56. Following ST-400 infusion, fetal hemoglobin levels have increased as compared to baseline and were continuing to rise as of the latest measurement of 2.8 g/dL at Day 90. Following an initial transfusion-free period of 7 weeks, the patient has received two PRBC transfusions commencing at 62 days post-infusion.

These studies and further studies of additional patients and patients 1-3 demonstrate that treatment of TDT including removing the need for additional therapies such as PRBC is achieved following administration (infusion) of genetically modified cells (ST-400) as described herein.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A genetically modified cell comprising a red blood cell (RBC) precursor cell comprising SB-mRENH1 mRNAs and SB-mRENH2 mRNAs, which mRNAs encode a ZFN pair; and
a genomic modification made following cleavage by the ZFN pair, wherein the modification is within an endogenous BCL11A enhancer sequence, such that the BCL11A gene is inactivated in the cell.

2. A composition comprising the genetically modified cells of claim 1 and cells descended therefrom.

3. An ex vivo method of treating a beta-thalassemia (β-thalassemia) in a subject in need thereof, the method comprising:
administering a composition according to claim 2 to the subject such that fetal hemoglobin (HbF) production in the subject is increased and one or more clinical symptoms of β-thalassemia are decreased, ameliorated, or eliminated.

4. The ex vivo method of claim 3, wherein the beta-thalassemia is transfusion-dependent β-thalassemia.

5. The ex vivo method of claim 3, wherein a change from baseline of clinical laboratory hemoglobin factions in grams/dL plasma and/or percent HbF of total hemoglobin (Hb) is achieved in the subject.

6. The ex vivo method of claim 3, wherein the hemoglobin factor is adult hemoglobin (HbA) and/or fetal hemoglobin (HbF).

7. The ex vivo method of claim 3, wherein the subject is β⁰/β⁰ or β⁰/β⁺.

8. The ex vivo method of claim 3, wherein levels of thalassemia-related disease biomarkers are altered following treatment.

9. The ex vivo method of claim 8, wherein the biomarkers are changes in iron metabolism; and/or changes in levels of erythropoietin, haptoglobin and/or hepcidin.

10. The ex vivo method of claim 3, wherein the clinical symptoms associated with iron overload or associated with baseline transfusion therapy are ameliorated or eliminated.

11. The ex vivo method of claim 10, wherein a decrease in endocrine dysfunction in the subject is assayed by determining levels and/or activity of thyroid hormones, IGF-1, morning cortisol, adrenocorticotropic hormone (ACTH), HbA1C, and/or vitamin D levels.

12. The ex vivo method of claim 3, wherein the need for RBC transfusions and infusion platelet transfusion, intravenous immunoglobin (IVIG) transfusion, plasma transfusion and/or granulocyte transfusion in the subject is(are) reduced or eliminated.

13. The ex vivo method of claim 3, wherein the clinical symptom reduced or eliminated in the subject is liver disease.

14. The ex vivo method of claim 3, wherein the clinical symptoms reduced or eliminated in the subject are cardiac abnormalities.

15. The ex vivo method of claim 3, wherein the clinical symptoms reduced or eliminated in the subject is/are osteoporosis and/or fractures.

16. The ex vivo method of claim 3, wherein baseline erythropoiesis is changed in the subject following administration of the composition.

17. The ex vivo method of claim 16, wherein hyperplasia is reduced or eliminated in the subject following administration of the composition.

18. The ex vivo method of claim 16, wherein the number of immature and/or cells with non-typical morphologies is/are reduced in the subject.

19. The ex vivo method of claim 3, wherein the number and percent of F cells in the subject is modified following administration of the composition.

20. The ex vivo method of claim 3, wherein the genetically modified cells are autologous or allogeneic.

21. The ex vivo method of claim 3, wherein the BCL11A-genetically modified cells further comprise one or more additional genetical modifications.

22. The ex vivo method of claim 21, wherein the genetically modified cells are allogeneic cells and the one or more additional genetic modifications comprise inactivation of one or more self-markers or antigens.

23. The ex vivo method of claim 3, wherein the genetically modified cells are hematopoietic stem cells isolated from the subject.

24. The ex vivo method of claim 23, wherein the hematopoietic stem cells are CD34+ hematopoietic stem or precursor cells (HSC/PC) and the CD34+ HSC/PC are mobilized in each subject by treatment with one or more doses of G-CSF and/or one or more doses of plerixafor prior to isolation.

25. The ex vivo method of claim 24, wherein at least 25×10⁶ CD34+ HSPCs/kg are mobilized in the subject and the mobilized cells are harvested by one or more apheresis cycles.

26. The ex vivo method of claim 3, further comprising, prior to administering the composition comprising the genetically modified cells to the subject and evaluating the cells of the composition for insertions and/or deletions within BCL11A.

27. The ex vivo method of claim 3, further comprising administering with one or more myeloablative condition agents one or more times to the subject prior to administration of the composition comprising the genetically modified cells.

28. The ex vivo method of claim 27, wherein the myeloablative agent comprises busulfan and further wherein:
intravenous (IV) administration of the busulfan is at between 0.5 to 5 mg/kg for one or more times;
IV administration of the busulfan is 3.2 mg/kg/day;
IV via central venous catheter for 4 days total dose of 12.8 mg/kg prior to infusion on Days −6 through −3 before infusion of the composition comprising the genetically modified cells on Day 0; or
IV administration of the busulfan is once daily or every 6 hours.

29. The ex vivo method of claim 3, wherein the dose of genetically modified cells administered to the subject is between 3×10⁶ cells/kg and 20×10⁶ cells/kg.

30. The ex vivo method of claim 3, wherein the genetically modified cells administered to the subject are formulated with approximately 1.0-2.0×10⁸ cells per bag at a concentration of approximately 1×10⁷ cells/mL.

31. The ex vivo method of claim 3, wherein the genetically modified cells are cryopreserved prior to administration and are administered to the subject within 15 minutes of thawing.

32. The ex vivo method of claim 3, further comprising monitoring the subject's vital signs prior to, during and/or after administration of the genetically modified cells.

33. The ex vivo method of claim 3, further comprising assessing hemoglobin, neutrophil and/or platelet levels in the subject prior to administration of the genetically modified cells to determine baseline levels of hemoglobin in the subject.

34. The ex vivo method of claim 33, wherein hemoglobin, neutrophil and/or platelet levels in the subject after administration of the genetically modified cells increase or remain stable as compared to baseline levels for weeks or months after administration.

35. The ex vivo method of claim 3, wherein the subject receives one or more packed red blood cell (PRBC)transfusions prior to and/or after administration of the genetically modified cells,

36. The ex vivo method of claim 3, wherein the need for additionally therapies such as a bone marrow transplant, blood component and/or iron chelation therapy PRBC transfusions in the subject are reduced or eliminated.

37. The ex vivo method of claim 36, wherein the need fir additional therapies is reduced or eliminated within 1-20 days of administration of the genetically modified cells.

38. The ex vivo method of claim 3, wherein the subject is monitored over time post administration to determine the indel profile of cells isolated from peripheral blood samples, bone marrow aspirates or other tissue sources in comparison with the indel profile of the infused cells to monitor stability of the graft in the subject.

39. The ex vivo method of claim 38, wherein the indel profile of the cells is monitored prior to administration to the subject.

40. An article of manufacture comprising a package comprising a composition according to claim 2 formulated in CryoStor® CS-10 cryomedia.

41. The article of manufacture of claim 40, wherein each bag contains approximately 1.0-2.0×10⁸ cells per bag at a concentration of approximately 1×10⁷ cells/mL.
